(19) United States
(12) Patent Application Publication
(10) Pub. No.: US 2006/0051859 A1
(43) Pub. Date: Mar. 9, 2006

(54) Long acting human interferon analogs

(76) Inventors: Yan Fu, Baltimore, MD (US); Zailin Yu, Baltimore, MD (US)

Correspondence Address: YU, Zailin;FortuneRock, Inc., 3120 SAINT PAUL STREET, Suite D109, BALTIMORE, MD, 21218, US

(21) Appl. No.: 10/936,447

(22) Filed: Sep. 9, 2004

(51) Int. Cl. C12N 15/74; C07H 21/04; C12N 1/18; C07K 14/56

(52) U.S. Cl. 435/320.1; 435/325; 435/254.2; 530/351; 536/23.5

(57) ABSTRACT

Compositions, kits and methods are provided for Interferon analogs in order to promote general health or for therapeutic treatment of diseases. Human interferon analogs are made by fusion of interferon with human serum albumin. The bio-assay shows that the interferon analogs with the same cell protection against viral attack have 3-10 times longer acting function than interferon in vivo. These novel long acting interferon analogs can be used in treatment of patients with viral infection, such as SARS virus, HIV, HCV, HBV, or HAV, and the cancer diseases, such as leukemia and malignant melanoma. They also have a 3-5 times longer shelf-life compared with interferon.

### CROSS REFERENCE TO RELATED APPLICATIONS

This application claims the priority benefit of U.S. Provisional Application Ser. No: 60/483,984 filed Jun. 30, 2003, which is hereby incorporated herein by reference in its entirety. Also, this invention is a continuation of U.S. Patent Provisional Application # 60/392,948 filed on Jul. 1, 2000 and U.S. patent application # 20040063635.

### BACKGROUND OF THE INVENTION

1. Field of the Invention

This invention relates to the manufacture and use of recombinant albumin fusion proteins to make human interferon analogs. The novel interferon analogs have the same functions with interferon in bio-assays, in vitro or in vivo. These long acting recombinant interferon analogs that are particularly expressed in yeast can largely improve interferon's therapeutic function.

2. Description of Related Art

1. Albumin

Albumin is a soluable, monomeric protein that comproses about one-half of the blood serum protein. Albumin functions primarily as a carrier protein for steroids, fatty acids, and thyroid hormones and plays a role in stabilizing extracellular fluid volume. Mutations in this gene on chromosome 4 result in various anomalous proteins. Albumin is a globular un-glycosylated serum protein of molecular weight 65,000. The human albumin gene is 16,961 nucleotides long from the putative ‘cap’ site to the first poly(A) addition site. It splits into 15 exons which are symmetrically placed within the 3 domains that are thought to have arisen by triplication of a single primordial domain. Albumin is synthesized in the liver as pre-pro-albumin which has an N-terminal peptide that is removed before the nascent protein is released from the rough endoplasmic reticulum. The product, proalbumin, is in turn cleaved in the Golgi vesicles to produce the secreted albumin. HSA has 35 cysteins; in blood this protein monomer has 17-disulfide linkage (Brown, J. R. “Albumin structure, Function, and Uses” Pergamon, N.Y., 1977). HSA is misfolded when produced intracellularly in yeast without its amino terminal secretion peptide sequence. This conclusion is based on its insolubility, loss of great than 90% of its antigenicity (as compared to human-derived HSA), and formation of large protein aggregates. At present albumin for clinical use is produced by extraction from human blood. The production of recombinant albumin in microorganisms has been disclosed in EP 330 451 and EP 361 991.

Albumin is a stable plasma transporter function provided by any albumin variant and in particular by human albumin. HSA is highly polymorphic and more than 30 different genetic alleles have been reported (Weikamp L, R, et al., Ann. Hum. Genet., 37 219-226, 1973). The albumin molecule, whose three-dimensional structure has been characterized by X-ray diffraction (Carter D. C. et al., Science 244, 1195-1198, 1989), was chosen to provide the stable transporter function because it is the most abundant plasma protein (40 g per liter in human), it has a high plasma half-life (14-20 days in human, Waldmann T. A., in “Albumin Structure, Function and Uses”, Rosenoer V. M. et al (eds), Pergamon Press, Oxford, 255-275,1977), and above all it has the advantage of being devoid of enzymatic function, thus permitting its therapeutic utilization at high dose.

2. Interferons

Interferons are a heterogeneous family of multifunctional cytokines whose first demonstrated biological activity was the induction of cellular resistance to virus infection. Antiviral activity of interferon was the only recognized biological function of the interferons for many years. Today interferons are found many other bio-functions. Interferon's actions on cell growth and differentiation and their many immunoregulatory activities are probably of greater fundamental biological significance.

Two very distinct families of proteins are counted among the interferons. The IFN-α/β “superfamily” (also called type I IFN) encompasses a group of structurally related genes and proteins that are further subdivided into the subfamilies IFN-α_{I} IFN-α/β, and IFN-β. The second “family” consists of a single gene encoding a single protein termed IFN-γ (also called type II IFN or immune IFN). It should be made clear at the outset that IFN-γ is structurally unrelated to the members of the IFN-α/β superfamily. The reasons for discussing IFN-α/β and IFN-γ together are largely historical. Interferon was first described by Isaacs and Lindenmnann (1957) as a product of virus-infected cells capable of inducing resistance to infection with homologous or heterologous viruses. A functionally related virus inhibitory protein (today termed IFN-γ) was described by Wheelock(1965) as an “Interferon-like” substance produced by mitogen-activated T-lymphocytes. For many years the only properties that made it possible to distinguish IFN-γ from the other interferons were its lack of stability at Ph 2 (Wheelcok 1965) and distinct antigenic specificity (Youngner and Salvin 1973). Only when the sequences of the proteins and genes of the major interferons were revealed in the early 1980s did it become clear what the relationship of the different interferons is to each other. People recognize now that IFN-γ is primarily an immunoregulatory cytokine whereas the potential actions of IFN-α/β extend to a broader variety of cells and tissues.

Members of the IFN-α/β superfamily represent the classical interferons. The first clear indication of the heterogeneity of the type I interferon proteins came from studies showing that interferons derived from human leukocytes and fibroblasts are antigenically distinct (Havell et al. 1975). Eventually leukocyte and fibroblast interferons were designated IFN-α and -β, respectively (COMMITTEE ON INTERFERON NOMENCLATURE 1980). Most of the information on interferon structure has been derived from gene cloning studies. At least 24 nonallelic human IFN-α genes and pseudogenes have been identified. They can be divided into two distinct subfamilies, termed IFN-α_{I} and -α_{II} (Weissmann and Weber 1986). The IFN-α_{I} subfamily potentially functional genes and several pseudogenes. The IFN-α_{II} subfamily is known to comprise only one functional gene and five or six nonallelic pseudogenes. IFN-αI genes encode mature proteins consisting of 165-166 amino acids; IFN-αII gene encodes a mature protein 172 amino acids long. All of the genes encode N-terminal secretive signal peptide presequences (generally 23 residues long) which are removed by proteolytic cleavage before the release of the mature interferon molecule from the cell. While it is clear that a high degree of homology is found among all human IFN-α genes and proteins, the IFN-α_{II} sequences have diverged significantly from the -α_{I} sequences, warranting their classification into a separate subfamily(Capon et al. 1985). In fact, it has been suggested that the IFN-α_{II} subfamily be named IFN-ω (Adolf 1987).

IFN-α forms vary in molecular mass between 19 and 26 kDa and are produced by monocytes/macrophages, lymphoblastoid cells, fibroblasts, and a number of different cell types following induction by viruses, nucleic acids, glucocorticoid hormones, and low-molecular weight substances. The effects of IFN-α are wide ranging and include potent anti-viral and anti-parasitic activity. In addition, IFN-α has anti-proliferative effects on certain tumor cells. Human IFN-α species lack potential N-glycosylation sites and most members of the IFN-α subfamilies in their native state are not glycosylated (Pestka 1983). Several natural human IFN-α proteins have been purified to homogeneity. They were shown to range in their apparent molecular weights from 16000 to 21000 (Rubinstein et al. 1981). The reason for these large differences in the apparent molecular weights has not been fully explained.

A single gene for human IFN-β encodes a 166-residue-long mature protein. Homology between IFN-β and members of the IFN-α_{I} subfamily is about 25-30% at the amino acid level and about 45% in the coding sequences at the nucleotide level(Taniguchi et al. 1980). In addition, there is also extensive homology in the 5′ nucleotide flanking regions which contain transcriptional promoter and enhancer sequences, reflecting the fact that IFN-α and -β genes are often coordinately induced (Degrave et al. 1981).

Interferons represent an important class of biopharmaceutical products, which have a proven track record in the treatment of a variety of medical conditions, including the treatment of certain autoimmune diseases, the treatment of particular cancers, and the enhancement of the immune response against infectious agents. To date, five types of interferons have been found in humans: interferon-alpha, interferon-beta, interferon-gamma, interferon-omega and a new form of human and murine interferon, “interferon-.epsilon.,” which have applications in diagnosis and therapy.

Interferon is used for treatment of Hepatitis C, B, and broad range of cancers, such as chronic myelogenous leukemia. Hepatitis C is an inflammation of the liver caused by hepatitis C virus infection. The HCV is most common chronic blood-borne disease in China (almost 80 millions HCV carrier) and USA (almost 4 millions HCV carriers), which causes 1 million people death worldwide per year. Chronic hepatitis B is an inflammation of the liver caused by HBV. The HBV infection can be developed into liver cancer and cirrhosis. 500 million people are infected by HBV in worldwide.

Production of IFN-α/β during virus infections is generally beneficial as it serves to limit the spread of virus and promote recovery (Gresser et al. 1976). In the past few years several types of interferon preparations have been licensed for clinical use. In the United States E. coli-derived recombinant human IFN-α 2 (IFN-α-2a) and IFN-α A (IFN-α-2b) have been approved for use in the treatment of hairy cell leukemia. IFN-α 2 and IFN-α A are both members of the IFN-α_{I} subfamily and they differ from each other in a single amino acid in position 23 (Arg in α 2 and Lys in α A). One of the preparations has also been approved for the treatment of condylomata acuminata. Other interferon preparations also have been approved for clinical use in some countries, e.g., a natural mixture of several IFN-α subtypes produced in the Namalwa line of human lymphoblastoid cells or natural human IFN-β produced in cultured fibroblasts. The approved use of these interferon preparations some countries includes chronic active hepatitis B, acute viral encephalitides, and nasopharyngeal carcinoma. A preparation of E. Coli-derived recombinant human IFN-γ has been approved for therapeutic use in rheumatoid arthritis in the German Federal Republic. Approved and experimental therapeutic applications of interferons have been extensively covered in a volume devoted to this topic (Finter and Oldham 1985). Interferon-beta, preferably in low doses, is used for stimulation of erythropoiesis in disorders characterized by lack of maturation of progenitor blood cells to red cells, (Michalevicz, U.S. Pat. No. 5,104,653)

Novel polypeptide produced by E. coli transformed with a newly isolated and characterized human IFN-.alpha and the gene is described. The polypeptide exhibits interferon activities such as antiviral activity, cell growth regulation, and regulation of production of cell-produced substances. Those novel interferon are named as Interferon-α-67, by Innis, in patent U.S. Pat. No. 5,098,703; Interferon-. alpha.54, in U.S. Pat. No. 4,975,276, and Interferon-.alpha.61, in U.S. Pat. No. 4,973,479.

Therapeutically synergistic mixtures of purified gamma interferon and purified interleukin-2 are provided for treatment of tumor-bearing hosts. Preferably, the gamma interferon and interleukin-2 are obtained from recombinant cell synthesis (Palladino U.S. Pat. No. 5,082,658).

The invention provides fusion proteins comprising an N-terminal region derived from an interferon-tau (IFN-.tau.) polypeptide and a C-terminal region derived from another type I interferon polypeptide, such as IFN-.alpha. or IFN-.beta. The fusion proteins exhibit reduced cytotoxicity compared to the corresponding unmodified type I interferons. Johnson, et al. U.S. Pat. No. 6,174,996 is the only patent that mentions how to make an interferon fusion protein.

A method that comprises administering a PEG.sub. 12000-IFN alpha conjugate to an individual afflicted with a viral infection susceptible of treatment with interferon alpha, preferably chronic hepatitis C, is disclosed. Glue et al. U.S. Pat. No. 5,908,621 is a patent mentions how to make a long acting or slow release form interferons. Shechter et al., (Proc. Natl. Acad. Sci. USA. Jan. 30, 2001; 98 (3): 1212-1217) reported the method to prolong the half-life of human interferon-α2 in circulation by covalently linked seven moieties of 2-sulfo-9-fluorenylmethoxycarbonyl (FMS) to the amino groups of human interferon-α2.

There is an invention that features a novel hybrid interferon species that comprises a chain of 161 and/or 162 amino acids. The hybrid is novel not only because its new structure, but also for the reason that the hybrid comprises a shortened or truncated segment of alpha interferon. Hence, an entirely new interferon species which does not occur in nature is reported by Leibowitz et al. in U.S. Pat. No. 4,892,743

Chang et al. in U.S. Pat. No. 5,723,125 patent disclosed a hybrid recombinant protein consisting of human interferon, preferably interferon-.alpha. (IFN.alpha.), and human immunoglobulin Fc fragment, preferably .gamma.4 chain. These two protein fragments are joined by a peptide linker comprising the sequence Gly Gly Ser Gly Gly Ser Gly Gly Gly Gly Ser Gly Gly Gly Gly Ser. This method makes an interferon-α fusion protein.

Kriegler, et al. in U.S. Pat. No. 5,324,655 patent reported a virion expression system for a desired protein packaged in an envelope derived from a retrovirus useful in administering proteins which cross cell membranes in order to serve their function. Preferred virions are those that carry an RNA sequence that encodes cytokines or lymphokines, and includes IL-2, multiple drug resistance protein, and TNF. Particularly disclosed is a DNA construct in which a gene encoding tumor necrosis factor (TNF) is directly linked to DNA encoding a human gamma-interferon signal peptide.

There are some research paper reported that the combination use of interferons could bring some beneficial to patients such as Trotta in U.S. Pat. No. 5,190,751 patent reported the human leukemia T-cells and B-cells are inhibited from proliferating by treatment with a combination of recombinant human alpha and gamma interferons, either simultaneously or sequentially, and the alpha interferon is preferably recombinant human alfa-2b interferon.

A common feature for any of these administration modes, however, is rapid inactivation of IFN-α in body fluids and in various tissues (O'Kelly, et al., 1985. Proc. Soc. Exp. Biol. Med. 178, 407-411). This in turn leads to the disappearance of the cytokine from the plasma within several hours after administration (Rostaing, et al., 1998, J Am. Soc. Nephrol. 9, 2344-2348). Unlike many other administered protein drugs, the major route of IFN-α elimination in vivo takes place in the circulatory system through proteolysis and inactivation by serum proteases. Therefore, long acting of interferon is needed in treatment of patients with viral infection or cancers in clinical trials.

### DETAILED DESCRIPTION OF THE INVENTION

The present invention provides innovative compositions, kits and methods for making long acting Interferon analogs in vivo that promote protection of virus infection and stimulate immune response to enhance general health or treat diseases or undesirable conditions.

In general, recombinant analog of interferon, fusion proteins of human serum albumin (HSA) and an Interferon, are provided in order to circumvent problems associated with conventional therapy using the Interferon protein itself. Generally, compared with the Interferon protein alone, the inventive Interferon analogs of the present invention possess the following advantages: 1) being capable of stimulating immune response of human body while viral infection happen; 2) allowing a slower release of the HSA-Interferon fusion in the body to maximize the therapeutic effects of the Interferon, and/or 3) reducing potential side effects or toxicity associated with administration of Interferon alone.

The present invention also provides a method for treating a patient with an Interferon in need thereof. In one embodiment, the method comprises administering a pharmaceutical formulation comprising an analog of Interferon to the patient in a therapeutically effective amount. The formulation may contain any pharmaceutically acceptable excipient and agents that stabilizes the HSA/IFN fusion protein. The formulation may further comprises natural or recombinant human serum albumin and/or another, different HSA/IFN fusion protein.

In addition, the present invention also provides efficient, cost-effective large scale production of these recombinant proteins in yeast. In particular, fusion proteins of HSA with each of human Interferon-α-2a, Interferon-α-2b, and Interferon-ω have been expressed in a yeast strain of Pichia pastoria and shown to have superior stability in storage and in plasma with the same bio-function in cell protection experiments in vitro.

### 1. HSA/IFN Fusion Proteins

In one aspect of the invention, isolated polynucleotides are provided that encode fusion proteins formed between HSA and an Interferon, i.e., HSA/IFN fusion. It should be noted that other types of albumin can also be employed to produce a fusion protein with an Interferon of the present invention.

The Interferon may include any protein that belongs to the family of Interferon. In a particular embodiment, the Interferon is a nature active cytokine produced by a virus infection. Examples of such a Interferon are described in Vilcek (1991) “Interferons”, in “Peptide Growth Factors and Their Receptors II”, edited by Sporn and Roberts, Spring-Verlag Heidelberg, New York Inc., USA.pp3-38 which is incorporated herein by reference in its entirety.

Specific examples of the Interferon include, but are not limited to, Interferon alpha -1 (IFNA-1), alpha-2 (IFNA-2), alpha-4 (IFNA-4), alpha-5 (IFNA-5), alpha-6 (IFNA-6), alpha-7 (IFNA-7), alpha-8 (IFNA-8), alpha-10 (IFNA-10), alpha-12 (IFNA-12), alpha-13 (IFNA-13), alpha-14 (IFNA-14), alpha-16 (IFNA-16), alpha-17 (IFNA-17), alpha-21 (IFNA2 1); Interferon-beta-1 (IFNB-1), interferon-beta-2 (IFNB-2, also be named as interleukin-6, IL-6); Interferon-lambda-1 (Interleukin-29), Interferon-lambda-2 (Interleukin-28A); and/or Interferon-epsilon.

Three distinct Interferon analogs have been made and well characterized: HSA-INF-α-2a, HSA-INF-α-2b, HSA-INF-β, HSA-INF-ω, and HSA-INF-γ. Other interferons or interferon family members are made by same techniques.

The Interferon may be linked directly to the N-terminus or C-terminus of HSA to form an analog. Optionally, there is a peptide linker (L) that links HSA and Interferon to form the fusion proteins HSA-L-IFN, or IFN-L-HSA. The length of peptide is usually between 2-100 aa ( preferably between 5-50 aa, and most preferably between 14-30 aa). The peptide linker may be a flexible linker that minimizes steric hindrance imposed by the bulk HA protein on inrterferon, such as a (G₄S)₃₋₄ linker. The linker addition may be good for interferon binds to its receptor. The addition of a linker to the in between of HSA and a therapeutic protein needs more work to validated the damage which may cause to when the fusion protein to be used as a therapeutic treatment on human. Because of the 6 amino acids and up peptides can have own immunity in human body. Preferably, there is no linker in the peptide of a human interferon analog. More preferably, there is no linker in the peptide of a long acting of HSA fusion protein drug.

The fusion protein may be a secret protein, which binds to a specific antibody of human albumin, and optionally, binds to a specific antibody of the interferon in this fusion protein.

In one embodiment, an isolated polynucleotide is provided that encodes a human serum albumin-interferon-α fusion protein (HSA-IFN-α-1β). The polynucleotide comprises a nucleotide sequence at least 90% identical to SEQ ID NO. 1 (FIG. 1). Preferably, the polynucleotide comprises a nucleotide sequence at least 95% identical to SEQ ID NO. 1. Preferably, the polynucleotide encodes an amino acid sequence comprising SEQ ID NO. 2 [HSA-IFN-α-1b].

In one embodiment, an isolated polynucleotide is provided that encodes a human serum albumin-interferon-α-2b fusion protein (HSA-IFN-α-2b). The polynucleotide comprises a nucleotide sequence at least 90% identical to SEQ ID NO. 3. Preferably, the polynucleotide comprises a nucleotide sequence at least 95% identical to SEQ ID NO. 3. Preferably, the polynucleotide encodes an amino acid sequence comprising SEQ ID NO. 4 [HSA-α-2b].

In another embodiment, an isolated polynucleotide is provided that encodes a human serum albumin-Interferon-β fusion protein (HSA-IFN-β). The polynucleotide comprises a nucleotide sequence at least 90% identical to SEQ ID NO. 5. Preferably, the polynucleotide comprises a nucleotide sequence at least 95% identical to SEQ ID NO. 5. Preferably, the polynucleotide encodes an amino acid sequence comprising SEQ ID NO. 6. [HSA-IFN-β].

In yet another embodiment, an isolated polynucleotide is provided that encodes a human serum albumin-Interferon-co fusion protein (HSA-IFN-ω). The polynucleotide comprises a nucleotide sequence at least 90% identical to SEQ ID NO.7. Preferably, the polynucleotide comprises a nucleotide sequence at least 95% identical to SEQ ID NO. 7. Preferably, the polynucleotide encodes an amino acid sequence comprising SEQ ID NO. 8 [HSA-IFN-ω].

In yet another embodiment, an isolated polynucleotide is provided that encodes a human serum albumin-Interferon-γ fusion protein (HSA-IFN-γ). The polynucleotide comprises a nucleotide sequence at least 90% identical to SEQ ID NO. 9. Preferably, the polynucleotide comprises a nucleotide sequence at least 95% identical to SEQ ID NO.9. Preferably, the polynucleotide encodes an amino acid sequence comprising SEQ ID NO. 10 [HSA-IFN-γ].

In yet another embodiment, an isolated polynucleotide is provided that encodes a human serum albumin-Interferon fusion protein (HSA-IFN). The polynucleotide comprises a nucleotide sequence at least 90% identical to SEQ ID NO. 11. Preferably, the polynucleotide comprises a nucleotide sequence at least 95% identical to SEQ ID NO. 11. Preferably, the polynucleotide encodes an amino acid sequence comprising SEQ ID NO. 12 [HSA]. Optionally, the polynucleotide further comprises a nucleotide sequence at least 90% identical to SEQ ID NOs. 13, 15, 17, 19, or 21. Preferably, the polynucleotide further comprises a nucleotide sequence encoding an amino acid sequence comprising SEQ ID NOs. 14, 16, 18, 20, or 22.

According to the embodiment, the Interferon may be selected from the group consisting, such as, but not limited, Interferon alpha-1 (IFNA-1), alpha-2 (IFNA-2), alpha-4 (IFNA-4), alpha-5 (IFNA-5), alpha-6 (IFNA-6), alpha-7 (IFNA-7), alpha-8 (IFNA-8), alpha-10 (IFNA-10), alpha-12 (IFNA-12), alpha-13 (IFNA-13), alpha-14 (IFNA-14), alpha-16 (IFNA-16), alpha-17 (IFNA-17), alpha-21 (IFNA21); Interferon-beta-1 (IFNB-1), interferon-beta-2 (IFNB-2, also be named as interleukin-6, IL-6); Interferon-lambda-1 (Interleukin-29), Interferon-lambda-2 (Interleukin-28A); and/or Interferon-epsilon.

The above-described polynucleotide with a sequence having a certain degree of sequence identity, for example at least 95% “identical” to a reference nucleotide sequence encoding a HSA/IFN fusion protein, is intended that the polynucleotide sequence is identical to the reference sequence except that the polynucleotide sequence may include up to five point mutations per each 100 nucleotides of the reference nucleotide sequence encoding the HSA/IFN fusion protein. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the polynucleotide sequence encoding a HSA/IFN fusion protein can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

When stored at ambient temperature or a lower temperature, the fusion protein of HSA and IFN may have a shelf-life 2 times longer, preferably 4 times longer, more preferably 6 times, and most preferably 10 times, longer than that of the IFN alone stored under the same condition.

The present invention involves the utilization of albumin as a vehicle to carry a therapeutic protein such as an IFN in the treatment of certain diseases such as cancers, or people in need of an increased blood cell proliferation in order to increase the blood cell numbers. The fusion protein of the present invention may be administered to a mammal, preferably a human, via a variety of routes, including but not limited to, orally, parenterally, intraperitoneally, intravenously, intraarterially, topically, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The analogs of Interferon, HSA-IFN, may also be delivered to the host locally (e.g., via stents or cathetors) and/or in a timed-release manner. In a particular embodiment, the fusion protein is delivered parenterally via injection.

When delivered in vivo to an animal, the fusion protein of HSA and IFN, Interferon analogs, may have a plasma half-life 2-10 times longer than that of the IFN alone.

The HSA/IFN fusion proteins of the present invention may also be administered in combination with a natural or recombinant human albumin, preferably a recombinant one at a therapeutically effective dose and ratio.

It is believed that after fusion with albumin, the IFN protein can have a longer shelf-life and plasma half-life, which allows cost-effective storage and transportation, as well as reduces amount and/or frequency of drug administration.

It is believed that other polypeptide form anti-virus or peptide inhibitors of virus entry cell after fusion with albumin, the peptide protein can have a longer shelf-life and plasma half-life, which allows maintaining same bio-function of peptide and gives a long acting therapeutic function. The peptides such as T20 can block the HIV virus entry of HIV targeted cells

### 2. Expression of Interferon Analogs in Host Organisms

The polynucleotides encoding the inventive Interferon analogs, HSA/IFN fusion proteins, can be cloned by recombinant techniques into vectors which are introduced to host cells where the fusion proteins can be expressed.

Generally, host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the polynucleotides encoding HSA/IFN fusion proteins. The culture conditions, such as temperature, pH and the like, are those previously used with the host cell selected for expression, and will be apparent to the ordinarily skilled artisan.

According to the invention, a recombinant vector is provided that comprises the polynucleotide sequence encoding an HSA/IFN fusion protein. The recombinant vectors can be an expression vector for expressing the Interferon analogs, HSA fusion protein encoded by the nucleic acid, HSA-IFN, HSA-L-IFN, or IFN-L-HSA in a host organism. The host organism includes, but is not limited to, mammalian (e.g., human, monkey, mouse, rabbit, etc.), fish, insect, plant, yeast, and bacterium.

Expression of the polynucleotide encoding an HSA/IFN fusion protein is under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, a tetracycline or tetracycline-like inducible promoter, the metallothionein promoter; heat shock promoters; the albumin promoter; the ApoAl promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters. The promoter also may be the native promoter which controls the polynucleotide encoding an HSA/IFN fusion protein.

Also according to the invention, a recombinant cell is provided that is capable of expressing comprises the polynucleotide sequence encoding an HSA/IFN fusion protein. The recombinant cell may constitutively or be induced in the presence or absence of an agent to express Interferon analog, HSA fusion protein, encoded by the nucleic acid, HSA-IFN, HSA-L-IFN, or IFN-L-HSA in a host organism. The type of the recombinant cell includes, but is not limited to, mammalian (e.g., human, monkey, mouse, rabbit, etc.), fish, insect, plant, yeast, and bacterial cell.

In a preferred embodiment, the host organism belongs to a genus of yeast such as Saccharomyces (e.g., S. cerevisiae), Pichia, Kluyveromyces, Hansenula, Torulaspora, and Schinosaccharomyces. In a more preferred embodiment, the host organism is Pichia pastoris. In a particular embodiment, the recombinant vector is a pPICZ A, pPICZ B, or pPICZ C.

Depending upon the host employed in a recombinant process for producing the fusion proteins, the fusion proteins of the present invention may be glycosylated or non-glycosylated. Preferably, when expressed in a host organism, the fusion protein of HSA and IFN may be glycosylated to substantially the same extent as that when expressed in mammalian cells such as Chinese hamster ovarian (CHO) cells, or as that when expressed in Pichia pastoris.

As indicated above, the albumin fusion proteins of the present invention are substantially preferably proteomic and can therefore be generated by the techniques of genetic engineering. The preferred way to obtain these fusion proteins is by the culture of cells transformed, transfected, or infected by vectors expressing the fusion protein. In particular, expression vectors capable of transforming yeasts, especially of the genus Pichia, for the secretion of proteins will be used.

It is particularly advantageous to express the HSA/IFN fusion protein in yeast. Such an expression system allows for production of high quantities of the fusion protein in a mature form, which is secreted into the culture medium, thus facilitating purification.

The development of yeast genetic engineering has been made possible the expression of heterologous genes and the secretion of their protein products from yeast. The advantages of protein secretion (export) of yeast include, but not limited to, high expression level, soluble protein, corrected folding, easy to scale-up and easy for purification.

HSA/IFN fusion proteins, the Interferon analogs, can be secreted into the media of yeast via an albumin natural secretion signal. The polypeptide sequence of HSA fusion protein can be preceded by a signal sequence which serves to direct the proteins into the secrete pathway. In a preferred embodiment the prepro-sequence of human albumin is used to secrete the fusion protein out of yeast cells into the culture medium. Other secrete signal peptides, such as the native Saccharomyces cerevisiae α-factor secretion signal, can also be used to make fusion protein of the present invention.

Yeast-expressed HSA is soluble and appears to have the same disulfide linkages as the human-blood derived counterpart. If used in a large scale production, which may be potentially used in gram amounts in humans, a recombinant HSA will require a close identity with the natural HSA product. Secreting the HSA/IFN fusion protein into the growth media of yeast, which is via prepro-amino-terminal processing (no initiator methionine residue), also circumvents the problems associated with preparing yeast extracts, such as the resistance of yeast cells to lysis. In addition, the purity of the product can be increased by placing the product in an environment in which 0.5-1.0% of total yeast proteins is included and the lacks toxic proteins that would contaminate the product.

In a preferred embodiment, a particular species of yeast Pichia pastoris is used in the system for expressing HSA/IFN fusions of the present invention. Pichia pastoris was developed into an expression system by scientists at Salk Institute Biotechnology/Industry Association (SIBA) and Phillips Petroleum for high-level expression of recombinant proteins. The techniques related to Pichia are taught in, for example, U.S. Pat. Nos.: 4,683,293, 4,808,537, and 4,857,467.

There are some advantages of using yeast Pichia pastoris to express HSA and HSA fusion proteins than using other systems. Pichia pastoris is a species of yeast genus, Pichia. Pichia has many advantages of higher eukaryotic expression systems such as protein processing, protein folding, and posttranslational modification, while it is as easy to manipulate as E. coli or Saccharomyces cerevisiae. It is faster, easier, and less expensive to use than other eukaryotic expression systems such as baculovirus or mammalian tissue culture, and generally gives higher expression levels. Pichia has an additional advantage which gives 10-100 fold higher heterogonous protein expression levels. Those features make Pichia a very useful protein expression system.

Due to the similarity between Pichia and Saccharomyces, many techniques developed for Saccharomyces may be applied to Pichia. These include transformation by complementation, gene disruption, and gene replacement. In addition, the genetic nomenclature used for Sac has been applied to Pichia. For example, histidinol dehydrogenase is encoded by HIS4 gene in both Sac and Pichia. Pichia as a methylotrophic yeast is capable of metabolizing methanol as its sole carbon source. The first step in the metabolism of methanol is oxidation of methanol to formaldehyde using molecular oxygen by the enzyme called alcohol oxidase. In addition to formaldehyde, this reaction also generates hydrogen peroxide. To avoid hydrogen peroxide toxicity, methanol metabolism takes place within a specialized cell organelle, called the peroxisome, which sequesters toxic by-products away from the rest of the cell. Alcohol oxidase has a poor affinity for O₂, and Pichia compensates it by generating large amounts of this enzyme. The promoter regulating the production of alcohol oxidase is the one used to drive heterogonous (HSA or HSA fused) protein expression in Pichia.

Compared with Saccharomyces cerevisiae, Pichia may have an advantage in glycosylation of secrete proteins because it generally does not hyper-glycosylate. Both Saccharomyces and Pichia have a majority of N-linked glycosylation of the high-mannose type; however, the length of the oligosaccharide chains that add post-translation ally to proteins in Pichia (average 8-14 mannose residues per side chain) is much shorter than those in Saccharomyces (50-150 mannose residues). Very little O-linked glycosylation has been observed in Pichia. In addition, Saccharomyces core oligosaccharide has terminal α-1,3 glycan linkages whereas Pichia does not. It is believed that the α-1,3 glycan linkages in glycosylated proteins produced from Saccharomyces are primarily responsible for the hyper-antigenic nature of those proteins that make them particularly unsuitable for therapeutic use. Although not yet proven, this is predicted to be less of a problem for glycoprotein generated in Pichia, because it may resemble the glycoprotein structure of higher eukaryotes. Protein expressed as a secrete form for correctly refolding and easy purification of HSA and HSA fusion proteins.

Watanabe, et al. (2001) “In vitro and in vivo properties of recombinant human serum albumin from Pichia pastoris purified by a method of short processing time”, Pharm Res 2001 Dec:18(12):1775; and Kobayashi, K et al. (1998) “The development of recombinant human serum albumin” Ther Apher, Nov:2(4):257-62.

There are many expression systems available for expressing in Pichia, such as EasySelect™ Pichia Expression Kit from Invitrogen, Inc. On this vector, an AOX1 promoter is used to allow methanol-inducible high level expression in Pichia and a Zeocin™ resistance as selective market for the recombinants from the transformation. Promoters (transcription initiation region) are very important in expressing fusion proteins in this invention.

AOX1 gene promoter is very strong in yeast system, especially in Pichia. Two Alcohol Oxidase Proteins are coded in Pichia for alcohol oxidase—AOX1 and AOX2. The AOX1 gene is responsible for the vast majority of alcohol oxidase activity in the cell. Expression of the AOX1 gene is tightly regulated and induced by methanol to very high levels, typically≧30% of the total soluble protein in cells grown with methanol as the carbon source. The AOX1 gene has been isolated and a plasmid-bone version of the AOX 1 promoter is used to drive expression of the gene of interest encoding the desired heterogonous protein (Ellis et al., 1985; Koutz et al., 1989; Tschopp et al., 1987a). While AOX2 is about 97% homologous to AOX1, growth on methanol is much slower than with AOX1. This slow growth on methanol allows isolation of Muts strains (aox1). Except for AOX1 gene promoter, other promoters can also be used to driver HSA fusion gene in yeast. They include the promoter from, but not limited to, PGK1, GAPDH, Ga11, Ga110, CYC1, PHO5, TRP1, ADH1, and ADH2 genes. In this invention, we also disclose a novel method to make recombinant yeast with dual expression cassette insertions at two separated locations.

The expression plasmid can also take the form of shuttle vectors between a bacterial host such as E. coli, DH5a from GIBCO/Life Science and yeast. The antibiotic Zeocin are used to be a marker for HSA carrier vector in all the examples.

The expression vector that contains the polynucleotide of HSA or HSA fusion therapeutic protein is introduced into yeast according to the protocols described in the kit from Invitrogen Inc. After being selected from transformed yeast colonies, those cells that express the HSA fusion protein of interest are inoculated into appropriate selective medium and then tested for their capacity to secrete the given fusion protein into the extracellular medium. The harvest of the protein can be conducted during cell growth for continuous cultures, or at the end of the growth phase for batch cultures. The fusion proteins which are the subject of this invention are then further purified from the culture supernatant by methods which take into account the albumin purification methods and pharmacological activities.

It is noted that other expression systems may also be used to express rHSA and HSA/IFN fusion proteins, including but not limited to, E. coli, B. Subtitis, Saccharomyces, Kluyveromyces, Hansenula, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis, animals, plants, and insect cells.

### 3. Combination Therapy of Interferon Analogs

The present invention also provides combinations of different Interferon analogs. The specific combinations of these interferon analogs or nature interferons may be administered to a patient to stimulate multiple types of protection to viral targeted cells or to synergistically enhance proliferation of a particular cell type. In particular, a combination of human albumin fusions with different hematopoietically active cytokines is used to effectively promote proliferation of the multiple blood cells and platelets. By using a combination of HSA/IFN fusion proteins targeting the signal transduction pathways of different types of blood cells, multiple blood functional cell production, such as platelets, erythrocytes and macrophages of white cells, can be increased after administration by just one injection.

In the present invention, the albumin's plasma transporter function and the therapeutic function of the IFN are integrated into a fusion form. The presence of albumin may confer a superior stability to the IFN by resisting degradation by proteases in the blood circulation, thus significantly prolonging the plasma half life of the IFN. Due to the masking effect of a bulky albumin, different IFNs fused with albumin in the combination may impose less interference with the biological function(s) of each other than a combination of the “naked” IFNs. Furthermore, an IFN fused with albumin may be slowly released in the system over an extensive period of time, thereby reducing the toxicity associated with injection of the IFN alone in abnormally high concentrations in the body. Such a slow release mode of action of the fusion protein combination can significantly reduce the amount and/or frequency of injections of the IFN, thereby further reducing the side effects of IFNs. Such combinations that are particularly useful for stimulating multiple blood cell proliferation after or before the chemo or radiation therapy of cancer patients who are tolerance for frequent, high dose injection of IFN are seriously compromised.

According to the present invention, HSA fusion protein with this type of IFN may remove above limitations by slowly releasing the drug into the patient's system. In addition, such fusion proteins may be combined with a relatively higher amount of albumin to further reduce the impact resulted from directly injecting the drug into the blood which causes a strong, adverse reaction of the central nervous system.

It is also known that “naked” cytokines (i.e., cytokines not fused to another protein such as HSA) are quite unstable when stored and have a short plasma half-life. Clearly, a therapeutic protein with such a weak stability in vivo constitutes a major handicap. In effect, repeated injections of the product, which are costly and inconvenient for patient, or an administration of product by perfusion, become necessary to attain an efficient concentration in plasma. Due to its extended plasma half and enhanced stability, the HSA/IFN fusion proteins of the present invention and their combinations, e.g., HSA fusions with Interferon-α, interferon-β, interferon-ω and interferon-γ, can be used to stimulate the production of antivirus peptides in plasma of humans.

In one embodiment, HSA/IFN-α fusion may be combined with HSA/IFN-γ fusion and the resulting combination may be administered to a patient with a virus infection to simultaneously stimulate secretion of antiviral peptides. For example, cancer patients may be injected with a combination of HSA/IFN-α and HSA/IFN-γ fusion proteins, before or after, a viral infection to avoid the damages of cells and organs. The Interferon-a will promoter the fight with virus and Interferon-g will fight inhibit the cancer cell proliferation.

Alternatively, an HSA/IFN fusion may be co-administered with a different HSA/IFN fusion simultaneously or sequentially to a patient in need thereof. This combination therapy may confer synergistic therapeutic effects on the patients. In one embodiment, the method is provided, comprising: administering a first pharmaceutical formulation comprising a first fusion protein of HSA and a first IFN to the patient in a therapeutically effective amount; and administering to the patient a second pharmaceutical formulation comprising a second fusion protein of HSA and a second IFN to the patient in a therapeutically effective amount. Such a combination therapy may confer synergistic therapeutic effects on the patient.

For example, HSA-IFN-α-2b fusion protein may be administered to the patient first, followed by administration of HSA-IFN-γ, HSA-IFN-ω and/or HSA-IFN-β at therapeutically effective doses and ratios to inhibit cancer cell proliferation of different and to induce antiviral peptide secretion from cells.

The present invention further provides a kit for use in the combination therapy described above. The kit comprises: a first fusion protein of HSA and a first IFN, and a second fusion protein of HSA and a second IFN. The first and second IFNs may be the same or different. For example, the first IFN is IFN-α-2b and the second IFN is Interferon-γ; the first IFN is Interferon-ω and the second IFN is Interferon-γ; or the first IFN is Interferon-β and the second IFN is Interferon-γ.

The HSA/IFN fusion proteins and their combinations thereof may be used to treat a wide variety of diseases, including but not limited to, the viral infection, such HAV, HBV, HCV, HPV, SARS virus, and/or HIV infection, tumors, cancers, renal failure, and tissue/organ transplantation. These fusion proteins are preferred not to contain non-human sequences that may elicit adverse immunogenicity in the patient. Interferon analogs are including but not limited to Interferon alpha-1 (IFNA-1), alpha-2 (IFNA-2), alpha-4 (IFNA-4), alpha-5 (IFNA-5), alpha-6 (IFNA-6), alpha-7 (IFNA-7), alpha-8 (IFNA-8), alpha-10 (IFNA-10), alpha-12 (IFNA-12), alpha-13 (IFNA-13), alpha-14 (IFNA-14), alpha-16 (IFNA-16), alpha-17 (IFNA-17), alpha-21 (IFNA21); Interferon-beta-1 (IFNB-1), interferon-beta-2 (IFNB-2, also be named as interleukin-6, IL-6); Interferon-lambda-1 (Interleukin-29), Interferon-lambda-2 (Interleukin-28A); and/or Interferon-epsilon.

### BRIEF DESCRIPTION OF THE FIGURES

FIG. 1 shows nucleotide and amino acid sequences of embodiments of Analogs of Interferon, HSA, and examples of individual IFNs.

FIG. 2 illustrates a plasmid DNA vector contains the HSA sequence and as a backbone vector for making Interferon analogs, HSA-IFN fusion proteins.

FIG. 3 shows a Western blot detected using mouse monoclonal anti-human serum albumin (Sigma Cat# A6684). Each lane was load with equivalent of 10 μl of culture medium supernatant from yeast after three-day expression. A), HSA (65Kd); B), Analog IFN-α-2a (84Kd); C). Analog IFN-β (84 kd); D). Analog IFN-ω (84kd); E). Control (yeast parent strain culture).

FIG. 4 shows a Western blot detected using Rabbit polyclonal anti-hIFN-α-2a antibody (Chemicon International Inc., Cat# Ab-218-NA), each lane contains 100 ng proteins. A), human IFN-α-2b (19 kd) expressed by E. coli; B), Analog Interferon-α 84 kd, HSA/IFN-α-2b fusion protein, expressed by yeast.

FIG. 5 is an Antiviral infection assay for human IFN-α and Analog Interferon-α, HSA/IFN-α-2a fusion protein, in WISH cell with VSV challenges.

FIG. 6 shows the results of a stability test of Interferon analog proteins under different temperature and its cell viral protection activity. A), 37° C.; B), 50° C.

FIG. 7 shows the long acting effects in vivo test of analog interferon, HSA-IFNs, in animal plasma, as compared with those when Interferon Analog or IFN were administered. A), 1-24hrs; B), 1-12 days.

### EXAMPLES

1. General Molecular Cloning Techniques

The classic methods of molecular cloning that include DNA preparative extractions, agarose and polyacrylamide electrophoresis, plasmid DNA purification by column or from gel, DNA fragment ligations, and restriction digestion are described in detail in Maniatis T. et al., “Molecular cloning, a Laboratory Manual”, Cold Spring Harbor laboratory, Cold Spring Harbor, N.Y., 1982 and will not be reiterated here.

Polymerase Chain Reaction (PCR) used through out all the examples is described by Saiki, R. K. et al, Science 230:1350-1354, 1985 and is carried out on a DNA thermal cycler (Perkin Elmer) according to the manufacturer's specification. DNA sequencing was performed by using standard facilities and following the method developed by Sanger et al., Proc. Natl. Acad. Sci. USA, 74:5463-5467, 1977. Oligonucleotides were synthesized by commercial facilities.

Transformation of E. coli was done by using DH5α competent cells from GIBCO/BRL. Qiagen plasmid DNA purification columns were used in the purification of plasmid DNAs. The transformation of yeast was carried out by electroporation following the instruction provided by the manufacturer or according to the manual of EasySelect™ Pichia Expression Kit (Invitrogen Inc). All yeast stains used in the examples are members of the family of Pichia, and in particular, the strain of Pichia pastoris (supplied by Invitrogen).

2. Construction of a Backbone Vector Expressing Human Serum Albumin

A total RNA isolated from human fetal liver was used in a reverse transcription polymerase chain reaction (RT-PCR) to generate the polynucleotide encoding human serum albumin. Briefly, 5 μg of RNA was reverse transcribed by adding a poly(T)_{18+N} primer and the SuperScript™ II RNase H reverse transcriptase (GIBCO/BRL) to make the complementary first strand of cDNA. The reaction was incubated at 45° C. for 20 minutes, then at 55° C. for 40 minutes.

The primers for cloning human serum albumin (HSA) are the following:
SEQ ID No. 23: 5′-GAATTCATGAAGTGGGTAACCTTTATTTCC-3′ and
SEQ ID No. 24: 5′-GAATTCTTATAAGCCTAAGGCAGCTTGACTTGC-3′.

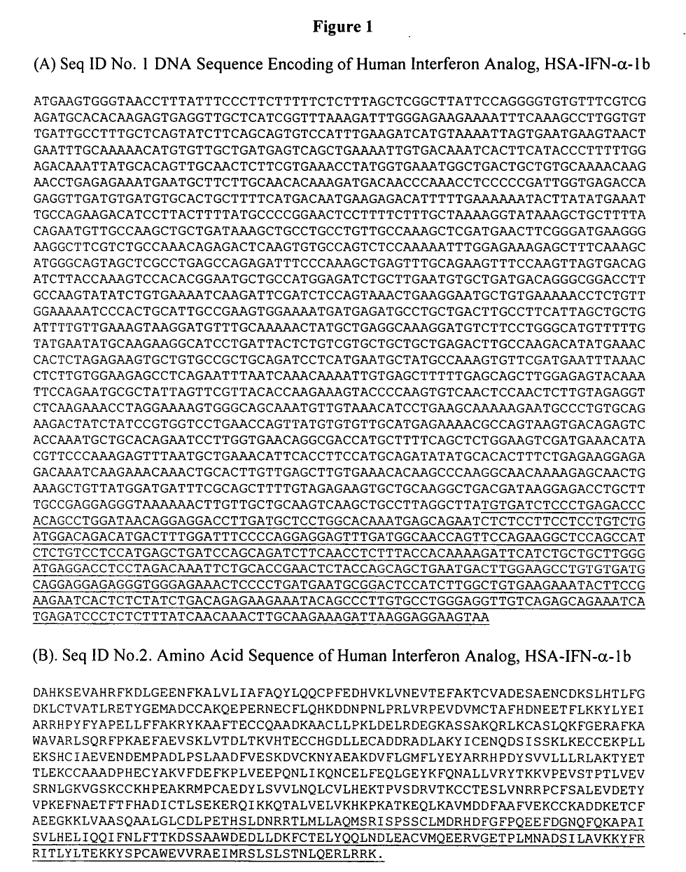

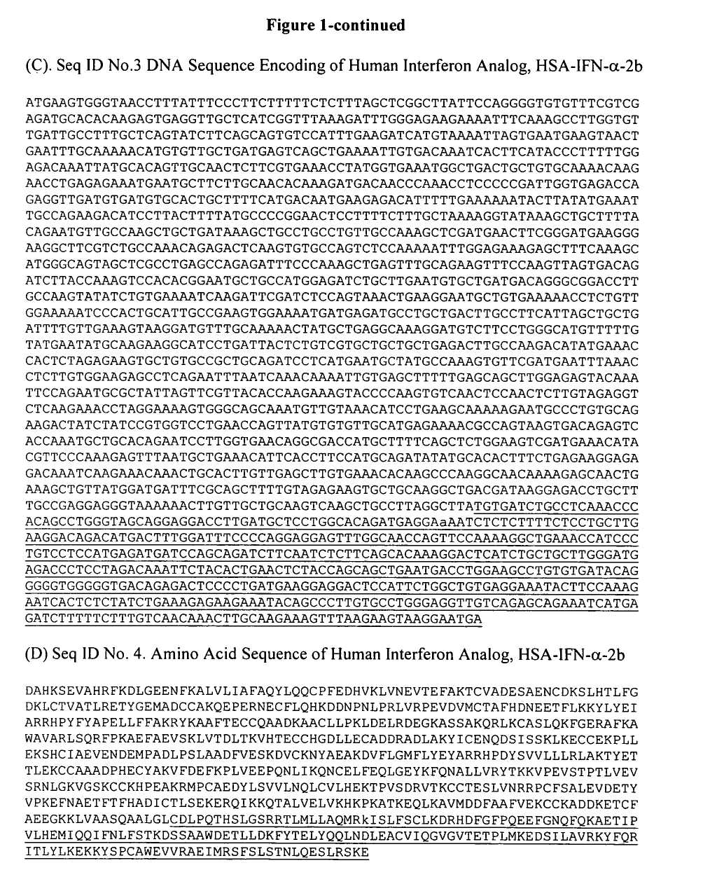

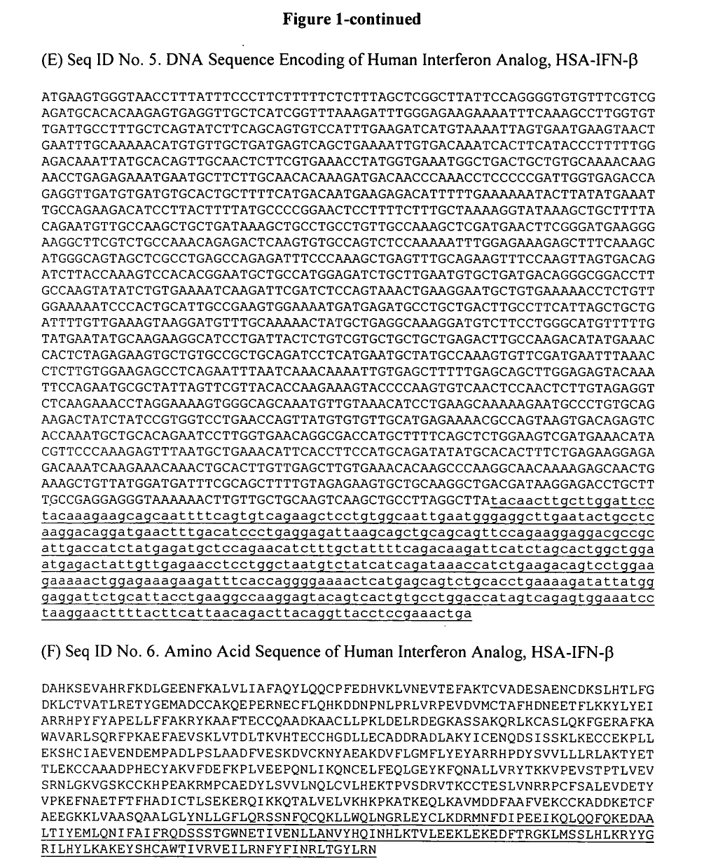

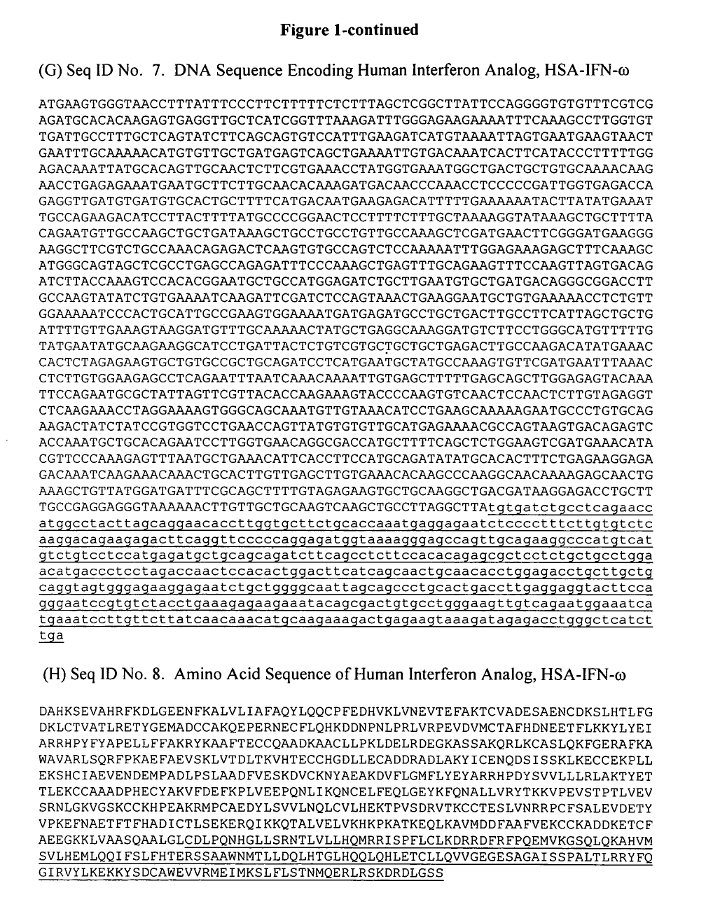

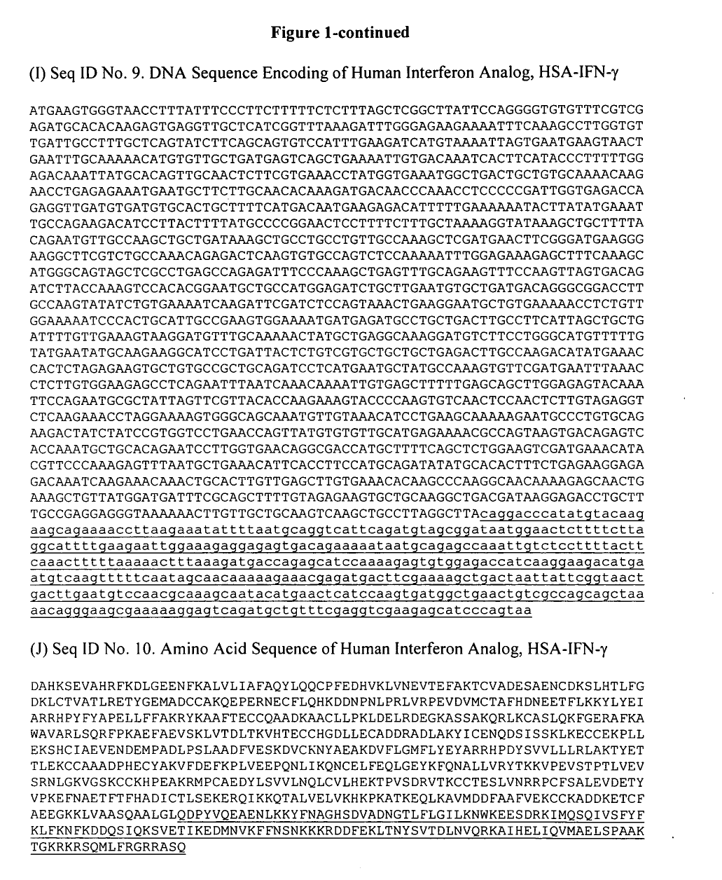

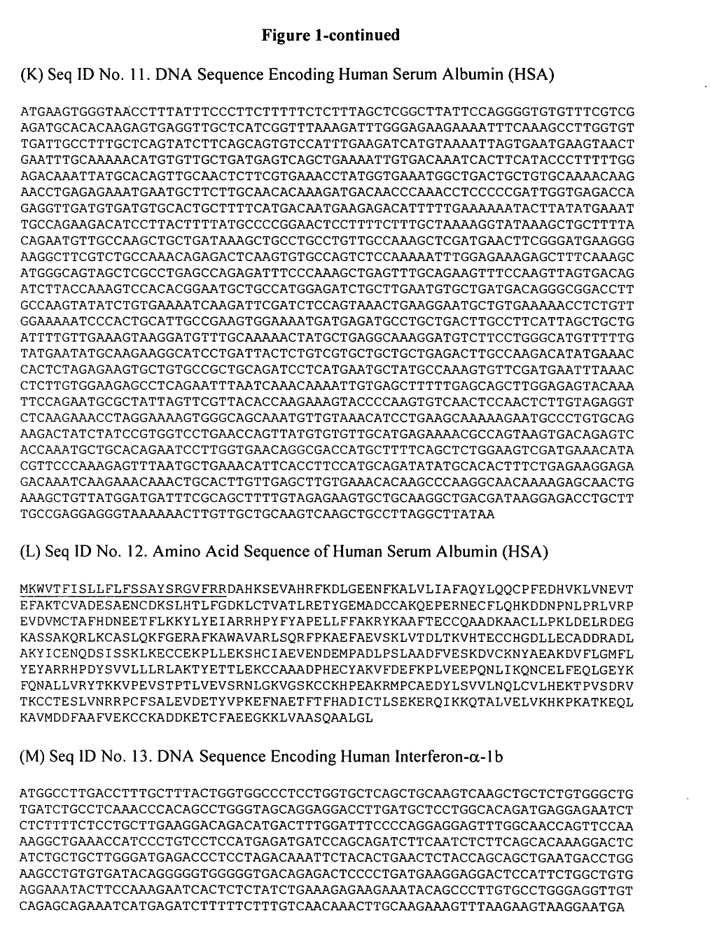

These primers were designed based on the HSA sequence published by GenBank (Access# V00494). Two EcoR I (underline of primers) sites were created at the 5′ end and 3′ end for sub-cloning into an expression vector. After inactivating the reverse transcriptase at 94° C. for 4 minutes, the DNA encoding of HSA was further amplified by Taq DNA PCR (Perkin Elmer) with 35 cycles of 94° C./30 seconds and 58° C./30 seconds and 72° C./2 minutes 30 second, followed by a 72° C./10 minutes incubation. The PCR product (1842 base pairs) was confirmed by 1% agarose gel electrophoresis. The product was subcloned into a pCR II TA cloning vector from Invitrogen. DNA sequencing confirmed that the plasmid DNA contained an insert whose polynucleotide sequence matches the DNA sequence published in GenBank (Access# V00494). FIG. 1, Seq ID No.11 is a polynucleotide DNA sequence and Seq ID No 12 is the protein amino acid sequence of human serum albumin.

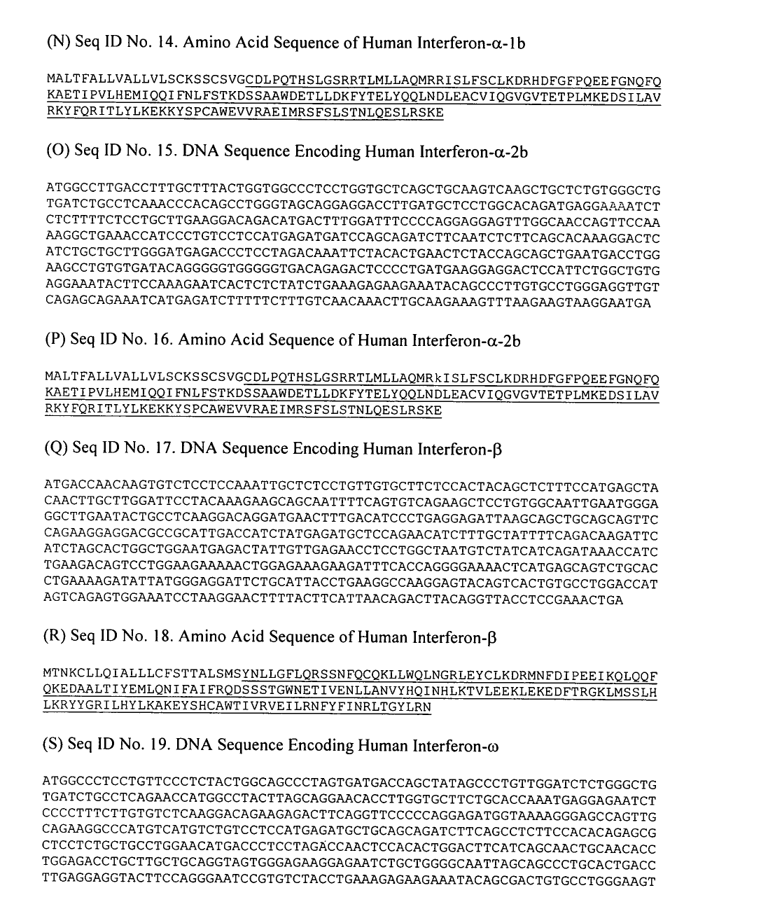

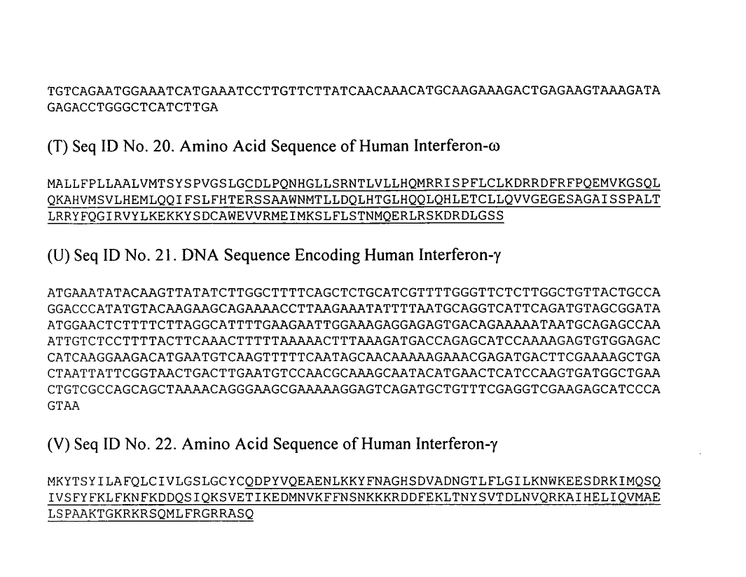

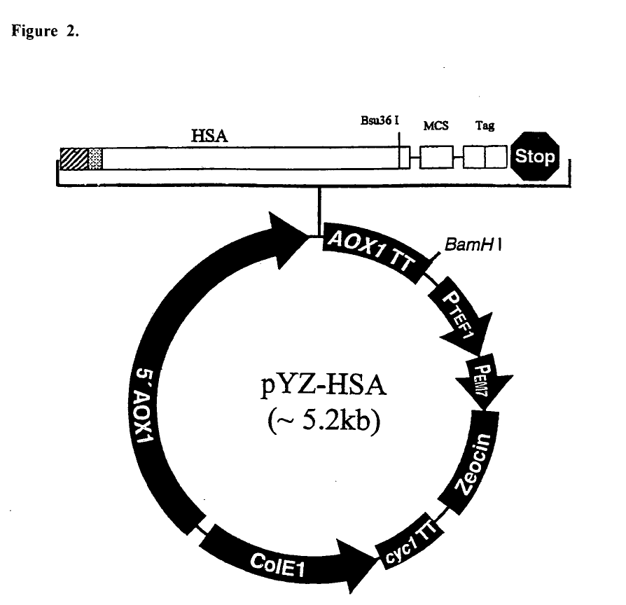

After restriction digestion of the PCR product with EcoR I, the gel purified HSA DNA fragment was inserted into the EcoR I site of a pPICZ-A or pGAPZ-A vector (provided by Invitrogen) or a new vector, pYH, modified by Zailin YU. After transformation of bacteria DH5α cells with this vector encoding HSA, a colony was selected from a low salt LB-agar plate contains 25 μg/ml Zeocin. The direction of the insert was confirmed by restriction enzyme double digestion of plasmid DNA by Xho I/Nde I. The constructs were designated as pYZ-HSA (Y: yeast vector; Z: Zeocin resistant) driven by AOX 1 or GAP promoter; or pYH-HSA (Y: yeast vector, Histidine resistant) driven by AOX1 or GAP (GAPDH) promoter and its physical maps are shown in FIG. 2.

There are some advantages associated with the vector constructed above. 1) It confers resistance to the antibiotic Zeocin. Zeocin is isolated from Streptomyces and is structurally related to bleomycin/phleomycin-type antibiotics. Antibiotics in the family of bleomycin/phleomycin are broad spectrum antibiotics that act as strong antibacterial and anti-tumor drugs. They show strong toxicity against bacteria, fungi (including yeast), plants, and mammalian cells. However, Zeocin is not as toxic as bleomycin on fungi. A single antibiotic Zeocin could be used in selecting the recombinants in both bacteria and in yeast. Further, there are multiple cloning sites at the 3′ end of HSA for conveniently subcloning an IFN protein in frame to encode a HSA-IFN. 2) A myc epitope sequence and a polyhistidine tag can be fused to the C-terminal of the expressed fusion protein for easy detection and/or purification by using commercially available antibodies against myc or polyhistidine tags. 3) AOX1 promoter or GAP promoter could be used which gives more choice for convenient expression of HSA/IFN. The GAP promoter is a no methanol inducer. By using of GAP promoter than AOX promoter, the industry scale level (1,000 Kg) fermentation would be safer with no use of methanol as an additive to induce the expression. 4) A dual expression cassette (promoter, to be expressed gene and resistant gene) from two vectors could be directly inserted with controlling into same yeast strain to make recombinant yeast for higher expression. Two vectors with promoter and insert, same or not, could be transformed into a yeast strain., pYZ-HSA, will directly insert at AOX1 gene locus with Zercin resistant, using same promoter's pYH-HSA, will directly insert at His gene location with His selction function. Vectors, pYH and pYZ as backbone vectors, were used in the construction of expression vectors for HSA fusion proteins described in the Example section.

3. Molecular Cloning of Human Interferons

3.1. Molecular Cloning OfHuman Interferon-α-1b Gene

Human Interferon-α-1b was cloned from a total RNA preparation of human white blood cells (monocytes/macrophages and B lymphocytes) by RT-PCR method described in Example 2. The oligonucleotide primers are

| | |
|---|---|
| SEQ ID NO. 25: | 5′-CATATGTGTGATCTCCCTGAGACCC-3′ |
| SEQ ID NO. 26: | 5′-GGATCCTTACTTCCTCCTTAATCTTTC-3′ |

A polynucleotide having 509 base pairs (bp) was amplified from RT-PCR reaction and subcloned into pCR II TA cloning vector from Invitrogen Inc. DNA sequencing confirmed the reading frame of human Interferon-α-1b. An Nde I restriction enzyme site was created at the 5′ end and a Bam HI site at the 3′ end (underline). The ATG initiate start codon of Interferon-a was included in this site (underlined in SEQ ID NO.25). The DNA sequence of human Interferon-α-1b (SEQ ID NO. 13) and its amino acid sequence (SEQ ID NO. 14) are shown in FIG. 1

3.2. Molecular Cloning Of Human Interferon-α-2a Gene

Human Interferon-α-2a was cloned from a total RNA preparation of human white blood cells (monocytes/macrophages and B lymphocytes) by RT-PCR method described in Example 2. The oligonucleotide primers are

| | |
|---|---|
| SEQ ID NO. 27: | 5′-CATATGGCCTTGACCTTTGCTTTAC-3′ |
| SEQ ID NO. 28: | 5′-GGATCCTCATTCCTTACTTCTTAAAC-3′ |

A polynucleotide having 579 base pairs (bp) was amplified from RT-PCR reaction and subcloned into pCR II TA cloning vector from Invitrogen Inc. DNA sequencing confirmed the reading frame of human Interferon-α-2a. An Nde I restriction enzyme site was created at the 5′ end and a Bam HI site at the 3′ end (underline). The ATG initiate start codon of Interferon-a was included in this site (underlined in SEQ ID NO. 27).

3.3. Molecular Cloning of Human Interferon-α-2b Gene

Human Interferon-α-2b gene has only one nucleatide different with Interferon-α-2a gene that result gives an amino acid different in position #23 (Arg in interferon-α-2a and Lys in interferon-α-2b). The interferon-α-2b gene was obtained by point mutation from cloned interferon-α-2a by a kit from Stratagene company. A paired mutation primers are used to make one nucleotide change in sequence. They are

SEQ ID NO. 29:
5′-TGGCACAGATGAGGAAAATCTCTCTTTTCTCCTGC-3′,
and
SEQ ID NO. 30:
5′-CAGGAGAAAAGAGAGATTTTCCTCATCTGTGCCAGC-3′.

The underlined nucleopeptide is the mutation point, from Interferon-α-2a, AGA (Arg) to Interferon-α-2b, AAA (Lys). The experiment was performed according to the manufacture's instruction. Mutated product in pCR II vector was sequence confirmed. The human Interferon-α-2b gene DNA sequence (SEQ ID NO. 15) and amino acid sequence (SEQ ID NO. 16) are showed in FIG. 1.

3.4. Molecular Cloning Of Human Interferon-β

Primers used to clone the human Interferon-β, gene from a cDNA library of human leukocyte are

| | |
|---|---|
| SEQ ID NO. 31: | 5′-CATATGACCAACAAGTGTCTCC-3′, and |
| SEQ ID NO. 32: | 5′-GAATTCTCAGTTTCGGAGGTAACC-3′ |

An Nde I site created at 5′ end and an EcoR I site at 3′ end of Interferon-β were created. The PCR products were gel-purified and subcloned into pCR2.1 TA cloning vectors and DNA sequence was confirmed. The human interferon-β DNA sequence (SEQ ID NO. 17) and the amino acid sequence (SEQ ID NO. 18) are shown in FIG. 1.

3.5. Molecular Cloning Of Human Interferon-ω

Human interferon-ω was cloned from a total RNA sample prepared from human cDNA Library of Leukocyte (White Blood Cells). The primers were:

| | |
|---|---|
| SEQ ID NO. 33: | 5′-CATATGGCCCTCCTGTTCCCTCTAC-3′, and |
| SEQ ID NO. 34: | 5′-GAATTCTCAAGATGAGCCCAGGTCTC-3′ |

The PCR products were gel-purified and inserted into pCR2.1 TA cloning vector and sequence confirmed. The human Interferon-ω DNA sequence (SEQ ID NO. 19) and amino acid sequence (SEQ ID NO. 20) are shown in FIG. 1.

3.6. Molecular cloning Of Human Interferon-γ

Human interferon-γ was cloned from a total RNA sample prepared from human cDNA library of mitogen-activated T-lymphocytes. The primers were:

| | |
|---|---|
| SEQ ID NO. 35: | 5′-CATATGAAATATACAAGTTATATC-3′ |
| SEQ ID NO. 36: | 5′-GAATTCTTACTGGGATGCTCTTCG-3′ |

The PCR products were gel-purified and inserted into pCR2.1 TA cloning vector and sequence confirmed. The human Interferon-γ DNA sequence (SEQ ID NO. 21) and amino acid sequence (SEQ ID NO. 22) are shown in FIG. 1.

4. In Frame Fusion of HSA With Human IFN-α-1b, IFN-α-2b, IFN-β, IFN-ω or IFN-γ

Interferon analogs were made by fusion human albumin gene with interferon gene. There is a Bsu36 I site at the C′-terminus of HSA. All of the Interferons described in the Example section were fused into this site by PCR primer extension to generate a restriction enzyme site of Bsu36 I at the N-terminus of the Interferon DNA sequence. The Interferon DNA fragments were amplified by PCR and then subcloned into Bsu36 I and Xho I sites of pYZ-HSA or pYH-HSA vector which had been double digested with Bsu36 I and Xho I to linearize the plasmid DNA.

4.1. Construction of Vector Containing Interferon Analogs, HSA/INF-α-1b

Interferon-α-1b was fused to HAS C′-terminus by using the following PCR primers:

SEQ ID NO. 37:
5′-CTGCCTTAGGCTTATGTGATCTCCCTGAGACCC-3′
and
SEQ ID NO. 38:
5′-TCTCGAGTTACTTCCTCCTTAATCTTTC-3′

(Human interferon-α-1b mature protein sequence is underlined in SEQ ID NO. 37).

A Xho I site (underlined in SEQ ID NO. 38) was created at the 3′ end of interferon-α-1b gene. The PCR products were digested with Bsu36I and Xho I, and the fragment was gel purified and inserted into pYZ-HSA or pYH-HSA between of Bsu36 I and Xho I sites to generate a new plasmid DNA, pYZ-HSA/IFN-α. The HSA-hIFN-α-1b hybrid polynucleotide sequence (SEQ ID NO. 1) and its fusion protein amino acid sequence (SEQ ID NO. 2) are showed in FIG. 1.

4.2. Construction of Vector Containing Interferon Analogs, HSA/INF-α-2a and HSA/IFN-α-2b

Interferon-α-2a or Interferon-a-2b gene was fused to HSA C′-terminus by using the following PCR primers:

SEQ ID NO. 39:
5′-CTGCCTTAGGCTTATGTGATCTGCCTCAAACCC-3′.

(Human Interferon-α-2a and Interferon-α-2b mature protein sequence is underlined), and

| | |
|---|---|
| SEQ ID NO. 40: | 5′-TCTCGAGTCATTCCTTACTTCTTAAAC-3′. |

A Xho I site (underlined in SEQ ID NO. 40) was created at the 3′ end of interferon-α gene. The PCR products were digested with Bsu36I and Xho I, and the fragment was gel purified and inserted into pYZ-HSA or pYH-HSA between of Bsu36 I and Xho I sites to generate a new plasmid DNA, pYZ-HSA/IFN-a. The HSA-hIFN-α-2b hybrid polynucleotide sequence (SEQ ID NO. 3) and its fusion protein amino acid sequence (SEQ ID NO. 4) are showed in FIG. 1.

4.3. Construction of Vector Containing Analog of Interferon-β, HSA/IFN-β

To make an analog of Interferon-β, HSA-IFN-β fusion protein, the following primers were designed SEQ ID NO. 41:

5′-CTGCCTTAGGCTTATACAACTTGCTTGGATTCC-3′ (human interferon-β mature protein sequence underlined), and SEQ ID NO. 42:

5′-CACTCGAGTCAGTTTCGGAGGTAACC-3′

(Xho I site underlined) and used to generate the modified human interferon-β DNA fragment. The PCR products were inserted between Bsu36I and Xho I sites of pYZ-HSA or pYH-HSA to generate a pYZ-HSA/IFN-β or pYH-HSA/IFN-β. The HSA-IFN-β hybrid polynucleotide sequence (SEQ ID NO. 5) and its fusion protein amino acid sequence (SEQ ID NO. 6) are shown in FIG. 1.

4.4. Construction of Vector Containing analog of Interferon-ω, HSA/IFN-ω

Human interferon-ω gene was fused with HSA DNA sequence by using two primers:

SEQ ID NO. 43:
5′-CTGCCTTAGGCTTATGTGATCTGCCTCAGAACCATGG-3′

(Interferon-ω mature protein sequence underlined), and

| | |
|---|---|
| SEQ ID NO. 44: | 5′-CTCGAGTCAAGATGAGCCCAGGTCTC-3′ |

(Xho I site at the 3′-teminus of interferon-ω underlined).

The PCR products were gel purified and subcloned between Bsu36I and Xho I sites of pYZ-HAS or pYH-HSA to generate a pYZ-HSA/IFN-w or pYH-HSA/IFN-ω. The analog of interferon-ω, HSA-INF-ω hybrid polynucleotide, sequence (SEQ ID NO. 7) and its amino acid sequence (SEQ ID NO. 8) are shown in FIG. 1.

4.5. Construction of Vector Containing Analog of interferon-γ, HSA/IFN-γ

The following primers:

SEQ ID NO. 45: 5′-ACTCCTTAGGCTTA CAGGACCCATATGTACAAGAAGC-3′ (Interferon-γ mature protein sequence underlined), and SEQ ID NO. 46: 5′-CTCGAGTTACTGGGATGCTCTTCG-3′ (Xho I site underlined) were used to modify Interferon-γ DNA sequence in order to subclone it into pYZ-HSA vector. PCR products were gel purified and double digested with Bsu36 I and Xho I and inserted between Bsu36 I and Xho I sites of pYZ-HSA, pYH-HSA to generate a pYZ-HSA/IFN-g, pYH-HSA/IFN-γ. The analog of Interferon-γ, HSA/IFN-γ hybrid polynucleotide, sequence (SEQ ID NO. 9) and its fusion protein amino acid sequence (SEQ ID NO.10) are shown in FIG. 1.

### 5. Transformation of Yeast

An expression cassette contains, a promoter driving of a gene, here is the analog of Interferon, a terminator, and a selective marker (such as Zeocin, antibiotic selection; Histidine, a deficient selection). Yeast strains, GS115, SMD1168 or ZY101 are Histidine synthesis deficiency. When transform the Yeast with the linearized yeast transfer shuttle vector, the expression cassette will be inserted directly to the location with a homologue region recombination. Most time one cassette will be inserted into a yeast host. In here, we disclosed a novel method for making of a dual insertion of expression cassette into a different chromosome region by two vectors with two different select markers.

5.1. Single Expression Cassette Insertion on Yeast A yeast Pichia pastoris strain, GS 115, colony was inoculated into 5 ml of YPD medium in a 50 ml conical tube at 30° C. overnight with shaking at 250 rpm. 0.2 ml of the culture was inoculated into 500 ml of YPD medium continually shaking at 30° C. for further 2-3 hours or until the cell density reach to OD₆₀₀=1.3-1.5. The cells were collected by centrifugation. The cell pellets were resuspend in 500 ml of ice-cold sterile water in order to wash the cells. After two rounds of washing, the cells were resuspended in 20 ml of ice-cold 1 M sorbitol to wash again and finally suspended in 1 ml of ice-cold 1 M sorbitol. The plasmid DNA constructs from Example 2, pYZ-HSA and in Example 4, pYZ-HSA/IFN-α-2a, pYZ-HSA/IFN-α-2b, pYZ-HSA/IFN-β, and pYZ-HSA/IFN-ω, pYZ-HSA/IFN-γ were linearized by PmeI restriction enzyme digestion first.

5 μg of each linear plasmid DNA was used to transform 80 μl of the freshly made yeast cells in an ice-cold 0.2 cm electroporation cuvette. The cells mixed with plasmid DNA were pulsed for 5-10 ms with field strength of 7500 V/cm. After the pulse, 1 ml of ice-cold 1 M sorbitol was immediately added into the cuvette and the content was transferred to a sterile 15 ml tube. The transformed cells were incubated in 30° C. without shaking for 2 hours then spread on pre-made YPD-agar plates with 100 μg/ml Zeocin. The colonies were identified with the insert and the expression level by SDS-PAGE or western-blot with proper antibodies. Different strains of Pichia, such as X-33, KM71 and proteinase deficient strain SMD1168, ZY10 (Constructed and be used in manufacture of recombinant secretory protein drugs by yeast system, Zailin YU unpublished data 2002) were tested for the expression and secretory of recombinant proteins.

5.2. Dual Expression Cassette Insertion on Yeast

In order to gain a higher expression level, people are trying to select multi-insertion from the recombinant yeast (Invitrogen Corp), But the select is no efficient, we use a second transformation method on a yeast is carrying an expression cassette. To do this, for example, we use pYZ-HSA/IFN-β transformed yeast, the HSA/IFN-β expression cassette has inserted at AOX1 Gene location in yeast chromosome with a Zeocin resistance, transformed again with pYH-HSA/IFN-β expression cassette by the method described in section of 5.1 again. The new select marker will be on the YPD-Agar plate contains no Histidine (His). Only the recombinant yeast contains the expression cassette with a Histidine gene can be survived in the medium. The new recombinant yeast now contains two genes of HSA/IFN-β, one located on AOX1 gene location, one is located on Histidinol dehydrogenase location. This recombinant yeast contains two selective markers and can grow in conditioned medium with antibiotic Zeocin, without the amino acid, Histidine, supplement.

By using this method, a different expression cassette also can be inserted to the yeast chromosome., such as the first expression cassette contains an interferon-a, and the second one is an interferon-γ; or the first expression cassette contains protein-X and the second expression cassette contains protein-X or protein different than first protein-X.

### 6. Secretion and Characterization of Interferon Analogs Expressed by Pichia

Several colonies from each transformation of the Interferon analog, HSA-IFN, were cultured with Zeocin in the buffered minimal medium with glycerol overnight or until OD₆₀₀=2−6 at 30° C. and shaking at 300 rpm. The cultured cells were collected by centrifuge at 1500 rpm for 5 minutes. Resuspend the cells into buffered minimal medium without glycerol and cell densities was keep in OD₆₀₀=1.0. 100% methanol was added into each flask to a final concentration at 0.5% every 24 hours to induce the protein expression. The culture medium was collected at different time points and the expression of each fusion protein was confirmed by SDS-PAGE and western blot. The results showed that human albumin and HSA-IFN fusion protein were expressed and secreted into the medium.

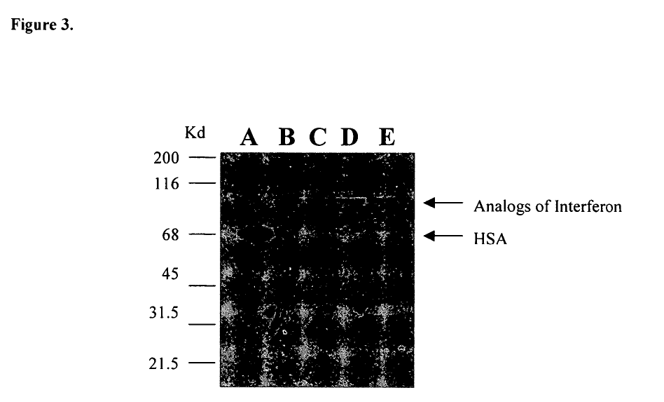

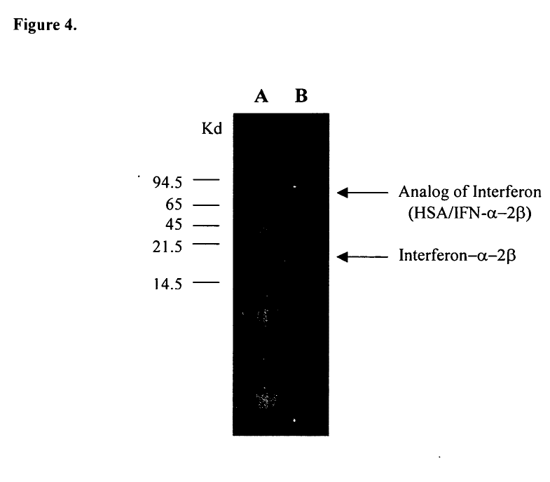

Mouse monoclonal anti-human serum albumin (Sigma) was used for immunoblotting on a SDS-PAGE gel. A typical Western blot experiment was carried on by electrophoresis transfer the protein from SDS-PAG to a nylon or nitrocellulose filter and incubated with a specific antibody (as the “first antibody”). Then an anti-first antibody would add to binding on the first antibody (as the “second antibody”). The second antibody was labeled with Fluorescence and the filter was exposed to an X-ray film. Protein molecular weight standard was used to determine the protein size. The results (FIG. 3) showed that the expressed recombinant proteins, HSA, Analog of Interferon-α (HSA-IFN-α-2a) therapeutic fusion protein, had an expected molecular weight and also had the same antigen as that of HSA prepared from a human blood plasma (Sigma). Using monoclonal anti-IFN-α specific antibody as first antibody, the HSA/Interferon-α fusion protein and human interferon-α (Chemicon International Inc. US) had the same antigen and showed that the molar ratio of HSA to interferon-α in the HAS/IFN-α-2a fusion protein is as expected (Zailin YU USPTO 60/392,948). Using monoclonal anti-Interferon-α specific antibody (CII, US) as first antibody, the HSA-IFN-α fusion protein and human Interferon-α (CII, US) had the same antigen and showed that the molar ratio of HSA to Interferon-β in the HSA/IFN-α fusion protein is as expected (FIG. 4).

7. Purification and Molecular Characterization of Interferon Analogs, HSA-IFNs

The cell culture medium (supernatant) containing the secreted protein of HSA or HSA-IFN fusion protein produced from the recombinant Pichia was collected, the salt concentration reduced, and the pH was adjusted to above 7.5. The concentrated sample was passed through an Affi-Gel Blue-gel (50-100 mesh) (Bio-Rad). The albumin or albumin fusion protein was bound to the matrix and eluded by a gradient 1-5 M NaCl. 75-85% pure albumin or albumin-IFN can be recovered in this step. If further purification is necessary, a size exclusion chromatography is applied to give a 95-99% purity of proteins. The pyrogen was removed from the protein samples in order to meet the requirement for use in in vivo test. The Affi-Prep Polymyxin Support (BIO-Rad) column was used to remove endotoxin from the samples. The purified protein finally passed through 0.2 μM filter to be sterilized and the protein concentration was measured by a standard method by using a Bio-Rad Protein Assay Kit.

8. Viral Protection Assay of Interferon Analog, Human Interferon-α-2a

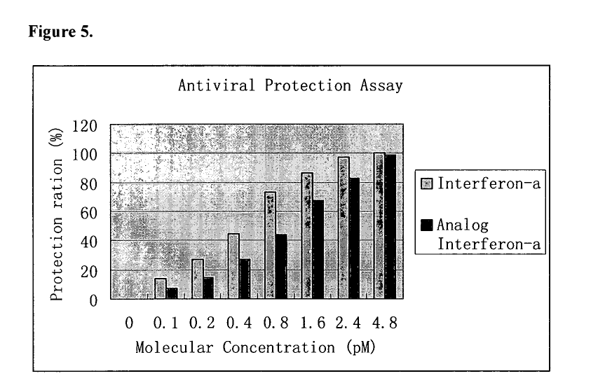

Antiviral activity of IFN-α-2a and its derivatives was determined by the capacity of the cytokine to protect human amnion WISH cells against vesicular stomatitis virus (VSV)-induced cytopathic effects (Rubinstein, et al., 1981, J virol. 37, 755-758). WISH cells (4.5×105 cells/ml) were seeded in a 96-well plate (100 μl/well) and incubated with 2-fold serial dilutions of IFN-α-2a or interferon analog, HSA/IFN-α-2a for 18 h at 37° C. WISH cell viability was determined by measuring the absorbance of crystal violet-stained cells in an ELISA plate. In this assay, native IFN-α-2a shows 50% protection of VSV-induced WISH cells (ED₅₀) at a concentration of 0.45±0.04 pM. The IFN-α-2a analog exhibiting ED₅₀ of 1.13±0.3 pM in this assay was considered as having 25% of the native antiviral potency (FIG. 5). Since HSA (65 kd) has a molecular weight about 3 times higher than that of interferon (19 kd), it can be inferred that HSA-IFN-α-2a fusion protein and Interferon analog have the same bioactivity as that of human Interferon-α-2a alone based on the molecular ratio.

9. Bioassay of Interferon-α analog, HSA/IFN-α, by ELISA

Enzyme-linked immunosorbent assay (ELISA) kit from Chemicon International, Inc. (California, US) was used for the quantitative determination of Interferon- concentration and bioactivities comparison with a commercial IFN-α sample. The IFN-α ELISA is based on the double-antibody sandwich method. With the ChemiKine™ assay system, pre-coated goat anti-rabbit antibody plates are used to capture a specific IFN-α complex in each sample consisting of IFN-α antibody, biotinylated IFN α, and sample/standard. The biotinylated IFN α conjugate (competitive ligand), and sample or standard compete for IFN α specific antibody binding sites. Therefore, as the concentration of IFN-α in the sample increases, the amount of biotinylated IFN α captured by the antibody decreases. The assay is visualized using a streptavidin alkaline phosphatase conjugate and an ensuing chromagenic substrate reaction. The amount of IFN α detected in each sample is compared to an IFN-α standard curve which demonstrates an inverse relationship between Optical Density (O.D.) and cytokine concentration: i.e. the higher the O.D. the lower the cytokine concentration in the sample. The amount of color generated was directly proportional to the amount of conjugate bound to the IFN-α antibody complex, which, in turn, was directly proportional to the amount of IFN-α in the protein samples or standard. The absorbance of this complex was measured and a standard curve was generated by plotting absorbance versus the concentration of the IFN-α standards. The IFN-α concentration of the unknown sample was determined by comparing the optical density of the protein samples to the standard curve. The standards used in this assay were recombinant human IFN-α (with kit) calibrated against the Second International Reference Preparation (67/343), a urine-derived form of human IFN-α. Human recombinant IFN-α expressed in CHO cells was used as a control to determine the rHSA/IFN-α bio-activity.

The results showed that the bioactivity of IFN-α fused to HSA had same activity compared with the native Interferon-α. When in a higher concentration of HSA-IFN-α in a sample, the size of HSA-IFN-α fusion protein molecule may be too large, which prevents the anti-IFN-α antibody from efficiently binding to the IFN-α molecule fused to HSA, thereby the sensitivity of the detection in this bioassay would be reduced. Same results were observed in HSA-EPO ELISA experiments (YU and FU, U.S. 20040063635).

10. Stability Testing of Interferon Analogs, HSA-IFNs Fusion Proteins In Vitro

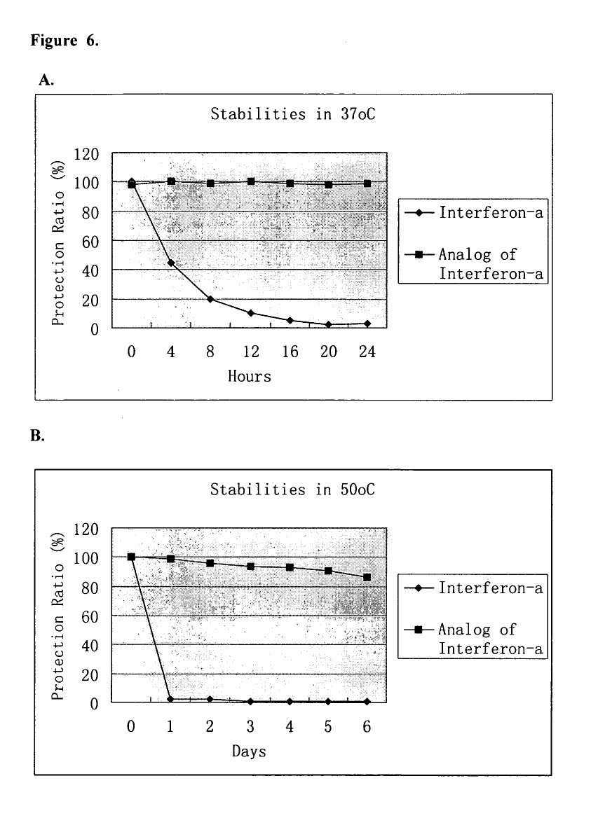

Using HSA/IFN-α-2a as an example, the stability of this interferon analog, HSA-interferons fusion protein, was tested at different time points at 37° C. and 50° C. 50 U (0.5 ng) of human interferon-α-2a from bacteria or 50 U (19.6 ng) of rHSA/IFN-α-2a was put into 200 μl thin-well PCR tube with 200 μl of tissue culture medium RPM1 without fetal bovine serum and other components. The tubes were sealed and left in water both. Samples were taken out at different time points and immediately put into −80 ° C. for storage. After all of samples were collected, a viral infection test on Wish cell line was carried out by standard protocols. The control of the test was set up in the same way as that in the bioassay. The results were showed that the “naked” human IFN-α lost almost all of its bioactivity after 10 hours at 37° C. (in FIG. 6 Panel A). But after 24 hours in 37° C., the bioactivity of Interferon Analog, HSA/IFN-α, still remained no changes. Experiment shows that even after 10 days, the antivirus potency has at least half remained. At 50° C. (Panel B), the “naked” human IFN-α lost its the bioactivity completely in 1 days. The Interferon Analog, HSA/IFN-α fusion protein, still retained near 90% of its bioactivity after 5 days. These results indicate that interferon analog may have a longer storage time and more resistant to degradation in harsh environment such as high temperatures.

11. Long Acting of Interferon Analogs in Plasma

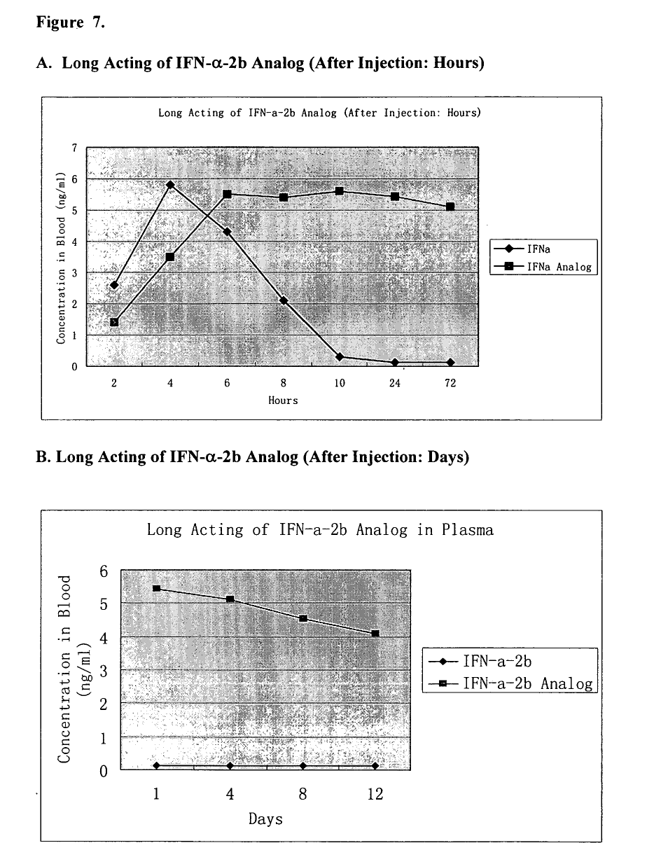

Human Interferon Analogs, interferon-α-2b (HSA/IFN-α-2b) and Interferon-α-2b, were tested for the long acting or slow release in animal in vivo. 15 ng (about 1×10³ U) human Interferon-α-2b plus 45 ng HSA (recombinant HAS from yeast) or 60 g (about 1×10³ U) human interferon-α-2b analog was injected into rats with 100 μl solution. After injection, the blood samples (0.05 ml) were collected. In the last day of experiments, a 05 ml of blood was collected from all the tested rats. The blood sample spin with EDTA added in a microcentrafuge tube. Blood supernatant was collected and storage at −80° C. Using Chemicon International, Inc. (California, USA) ChemiKine™ Human IFNα EIA Kit (Cat# CYT102) tested all the blood samples from the rat injected with interferon-α-2b (control) and Interferon Analog, HSA/IFN-α-2b. The results showed that Interferon analog maintains much longer undigested status in plasma than the “naked” interferon-α-2b even with same amount of HSA injection (FIG. 7). The interferon-α-2b only can be detected from plasma in about 10 hours. The Interferon analog, HSA/IFN-α-2b can be detected even after 12 days of injection. This results also match to the report that albumin has a half-life in plasma about 20 days (Waldmann T. A., in “Albumin Structure, Function and Uses”, Rosenoer V. M. et al (eds), Pergamon Press, Oxford, 255-275,1977). The novel form interferon shows a greater half-life in plasma. The plasma samples at day 12 were tested for their antiviral protection to WISH cells. The results showed that the control sample has no antiviral protection bio-function, but Interferon analog still maintains some bio-function in viral protection to the tested cells. This long acting bio-function gives Interferon analogs novel utilities when be used in clinical and therapeutic to patients as a recombinant protein drugs.

12. Expression and Scale-Up of Interferon Analogs by Fermentation

In this example, it is shown that expression and scale-up are much easier by using a Pichia system than other surrently available system. After Pichia recombinants were isolated, expression of both Mut+ and Mut⁵ recombinants was tested. This involved growing a small culture of each recombinant, inducing with methanol, and taking sample at different time points. For secrete expression, both the cell pellet and supernatant were analyzed from each time point. The samples were analyzed on SDS-PAGE gels by using both Coomassie staining and Western blot. Bioactivities of expressed samples were tested and the expression levels and purity were monitored in each step for production of HSA fusion proteins.

### References

Brown, J. R. “Albumin structure, Function, and Uses” Pergamon, N.Y. 1977
Weikamp L, R, et al., Ann. Hum. Genet., 37 219-226, 1973
Carter D. C. et al., Science 244, 1195-1198, 1989
Waldmann T. A., in “Albumin Structure, Function, and Uses”, Rosenoer V. M. et al (eds), Pergamon Press, Oxford, 255-275, 1975
Shechter et al., Proc. Natl. Acad. Sci. USA. 2001 January 30; 98 (3): 1212-1217
O'Kelly, et al., 1985. Proc. Soc. Exp. Biol. Med. 178,407-411
Rostaing, et al., 1998, J Am. Soc. Nephrol. 9, 2344-2348
Vilcek (1991) “Interferons”, in “Peptide Growth Factors and Their Receptors II”, edited by Sporn and Roberts, Spring-Verlag Heidelberg, New York Inc., USA.pp3-38

### Patent References

EP 330 451
EP 361 991
U.S. Pat. No. 5,098,703
U.S. Pat. No. 4,973,479.
U.S. Pat. No. 4,975,276
U.S. Pat. No. 5,082,658
U.S. Pat. No. 6,174,996
U.S. Pat. No. 5,908,621
U.S. Pat. No. 4,892,743
U.S. Pat. No. 5,723,125
U.S. Pat. No. 5,324,655
U.S. Pat. No. 5, 190,751

What is claimed are:

1. An isolated polynucleotide encoding an interferon analog protein formed between a human serum albumin (HSA) and an Interferon (IFN), comprising: a first nucleotide sequence at least 90% identical to SEQ ID NO. 11 and a second nucleotide sequence encoding an IFN positioned either 5′- or 3′- to the first nucleotide sequence, wherein the first and second nucleotide sequences are operably linked to be expressed as a fusion protein of HSA and IFN.

2. The isolated polynucleotide of claim 1, wherein the first nucleotide sequence is at least 95% identical to SEQ ID NO. 11 and, wherein the first nucleotide sequence encodes an amino acid sequence comprising SEQ ID NO. 12.

3. The isolated polynucleotide of claim 1, wherein the second nucleotide sequence is at least 95% identical to SEQ ID NO. 13, 15, 17, 19, or 21 and, wherein the second nucleotide encodes an amino acid sequence comprising SEQ ID NO. 14, 16, 18, 20, or 22.

4. The isolated polynucleotide of claim 1, wherein the IFN is selected from the group consisting of Interferon alpha-1 (IFNA-1), alpha-2 (IFNA-2), alpha-4 (IFNA-4), alpha-5 (IFNA-5), alpha-6 (IFNA-6), alpha-7 (IFNA-7), alpha-8 (IFNA-8), alpha-10 (IFNA-10), alpha-12 (IFNA-12), alpha-13 (IFNA-13), alpha-14 (IFNA-14), alpha-16 (IFNA-16), alpha-17 (IFNA-17), alpha-21 (IFNA2 1); Interferon-beta-1 (IFNB-1), interferon-beta-2 (IFNB-2, also be named as interleukin-6, IL-6); Interferon-lambda-1 (Interleukin-29), Interferon-lambda-2 (Interleukin-28A); and/or Interferon-epsilon.

5. The isolated polynucleotide of claim 1, wherein the protein encoded by polynucleotide binds to a specific antibody of human albumin.

6. A recombinant vector, comprising: the sequence of the polynucleotide in claim 1,

7. A recombinant cell containing the recombinant vector of claim 6.

8. The recombinant vector of claim 6, wherein the vector is an expression vector for expressing the fusion protein in a host organism selected from the group consisting of mammal, fish, insect, plant, yeast, and bacterium.

9. The recombinant vector of claim 7, wherein the host organism is yeast and the yeast is selected from the group consisting of, but not limited, Saccharomyces, Hansenula, Canadida, Pichia, Kluyveromyces, Torulaspora, or Schinosaccharomyces.

10. The isolated polynucleotide of claim 1, further comprising a third nucleotide sequence encoding a peptide linker that links the HSA and the IFN.

11. The third polynucleotide of claim 10, wherein the length of the peptide linker is 2-50 aa, preferable, wherein the peptide linker is a (G₄S)₃₋₄ linker.

12. A recombinant protein having an amino acid sequence selected from the group consisting of SEQ ID NOs: 2, 4, 6, 8, and 10.

13. The recombinant protein of claim 12, wherein the protein is recombinantly produced in yeast cells and glycosylated to substantially the same extent as that when recombinantly produced in mammalian cells.

14. The recombinant protein of claim 12, wherein the mammalian cells are CHO cells and wherein the yeast cells are Pichia pastoris cells.

15. The recombinant protein of claim 12, wherein the protein has a shelf-life at least 5 times longer than that of the IFN alone when stored under the same condition.

16. The recombinant protein of claim 12, wherein the protein has a plasma half-life at least 3 times longer than that of the IFN alone when administered in vivo.

17. A composition, comprising: a combination of at least two different interferon analogs, HSA/IFN fusion proteins.

18. The composition of claim 17, wherein the combination is HSA/IFN-α and HSA/IFN-γ interferon analogs, wherein the combination is HSA/IFN-α and HSA/IFN-β interferon analogs, or wherein the combination is interferon analog, HSA/IFN-γ, and interferon analog, HSA/IFN-ω.

19. A method for treating a patient with an IFN in need thereof, comprising: administering a pharmaceutical formulation comprising a fusion protein of HSA and IFN to the patient in a therapeutically effective amount.

20. A method for treating a patient with a hematological disorder, comprising: administering a first pharmaceutical formulation comprising a first fusion protein of HSA and a first IFN to the patient in a therapeutically effective amount; and administering to the patient a second pharmaceutical formulation comprising a second fusion protein of HSA and a second IFN to the patient in a therapeutically effective amount.

21. A method for treating a patient with a hematological disorder, comprising: administering the composition of claim 17 to the patient in a therapeutically effective amount.

22. A kit, comprising: a first fusion protein of HSA and a first IFN, and a second fusion protein of HSA and a second IFN.

23. The kit of claim 22, wherein the first and second IFN are different.


SEQUENCE LISTING (46 sequences)

SEQ ID NO: 1 - DNA, 2325 bp, Artificial
  Synthesis
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaagtaa

SEQ ID NO: 2 - protein, 750 aa, Artificial
  Synthesis
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPEVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRK

SEQ ID NO: 3 - DNA, 2325 bp, Artificial
  Synthess
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgtgatctgcctcaaacccacagcctgggtagcaggaggaccttgatgctcctggcacagatgaggaaaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 4 - protein, 750 aa, Artificial
  Synthesis
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPEVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 5 - DNA, 2322 bp, Artificial
  Synthesis
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga

SEQ ID NO: 6 - protein, 749 aa, Artificial
  Synthesis
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPEVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 7 - DNA, 2346 bp, Artificial
  Synthesis
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgtgatctgcctcagaaccatggcctacttagcaggaacaccttggtgcttctgcaccaaatgaggagaatctcccctttcttgtgtctcaaggacagaagagacttcaggttcccccaggagatggtaaaagggagccagttgcagaaggcccatgtcatgtctgtcctccatgagatgctgcagcagatcttcagcctcttccacacagagcgctcctctgctgcctggaacatgaccctcctagaccaactccacactggacttcatcagcaactgcaacacctggagacctgcttgctgcaggtagtgggagaaggagaatctgctggggcaattagcagccctgcactgaccttgaggaggtacttccagggaatccgtgtctacctgaaagagaagaaatacagcgactgtgcctgggaagttgtcagaatggaaatcatgaaatccttgttcttatcaacaaacatgcaagaaagactgagaagtaaagatagagacctgggctcatcttga

SEQ ID NO: 8 - protein, 757 aa, Artificial [sequence omitted]

SEQ ID NO: 9 - DNA, 2259 bp, Artificial
  synthesis
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttacaggacccatatgtacaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 10 - protein, 728 aa, Artificial
  synthesis
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPEVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLQDPYVQEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 11 - DNA, 1830 bp, Homo sapiens
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 12 - protein, 585 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPEVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 13 - DNA, 567 bp, Homo sapiens
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 14 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 15 - DNA, 567 bp, Homo sapiens
atggccttgacctttgctttactggtggccctcctggtgctcagctgcaagtcaagctgctctgtgggctgtgatctgcctcaaacccacagcctgggtagcaggaggaccttgatgctcctggcacagatgaggaaaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 16 - protein, 188 aa, Homo sapiens [sequence omitted]

SEQ ID NO: 17 - DNA, 564 bp, Homo sapiens
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga

SEQ ID NO: 18 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 19 - DNA, 588 bp, Homo sapiens
atggccctcctgttccctctactggcagccctagtgatgaccagctatagccctgttggatctctgggctgtgatctgcctcagaaccatggcctacttagcaggaacaccttggtgcttctgcaccaaatgaggagaatctcccctttcttgtgtctcaaggacagaagagacttcaggttcccccaggagatggtaaaagggagccagttgcagaaggcccatgtcatgtctgtcctccatgagatgctgcagcagatcttcagcctcttccacacagagcgctcctctgctgcctggaacatgaccctcctagaccaactccacactggacttcatcagcaactgcaacacctggagacctgcttgctgcaggtagtgggagaaggagaatctgctggggcaattagcagccctgcactgaccttgaggaggtacttccagggaatccgtgtctacctgaaagagaagaaatacagcgactgtgcctgggaagttgtcagaatggaaatcatgaaatccttgttcttatcaacaaacatgcaagaaagactgagaagtaaagatagagacctgggctcatcttga

SEQ ID NO: 20 - protein, 194 aa, Homo sapiens
ALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 21 - DNA, 501 bp, Homo sapiens
atgaaatatacaagttatatcttggcttttcagctctgcatcgttttgggttctcttggctgttactgccaggacccatatgtacaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 22 - protein, 166 aa, Homo sapiens [sequence omitted]

SEQ ID NO: 23 - DNA, 30 bp, Artificial
  synthesis
gaattcatgaagtgggtaacctttatttcc

SEQ ID NO: 24 - DNA, 25 bp, Artificial
  synthesis
catatgtgtgatctccctgagaccc

SEQ ID NO: 25 - DNA, 25 bp, Artificial
  synthesis
catatgtgtgatctccctgagaccc

SEQ ID NO: 26 - DNA, 27 bp, Artificial
  synthesis
ggatccttacttcctccttaatctttc

SEQ ID NO: 27 - DNA, 25 bp, Artificial
  synthesis
catatggccttgacctttgctttac

SEQ ID NO: 28 - DNA, 26 bp, Artificial
  synthesis
ggatcctcattccttacttcttaaac

SEQ ID NO: 29 - DNA, 35 bp, Artificial
  synthesis
tggcacagatgaggaaaatctctcttttctcctgc

SEQ ID NO: 30 - DNA, 36 bp, Artificial
  synthesis
caggagaaaagagagattttcctcatctgtgccagc

SEQ ID NO: 31 - DNA, 22 bp, Artificial
  synthesis
catatgaccaacaagtgtctcc

SEQ ID NO: 32 - DNA, 24 bp, Artificial
  synthesis
gaattctcagtttcggaggtaacc

SEQ ID NO: 33 - DNA, 25 bp, Artificial
  synthesis
catatggccctcctgttccctctac

SEQ ID NO: 34 - DNA, 26 bp, Artificial
  synthesis
gaattctcaagatgagcccaggtctc

SEQ ID NO: 35 - DNA, 24 bp, Artificial
  synthesis
catatgaaatatacaagttatatc

SEQ ID NO: 36 - DNA, 24 bp, Artificial
  synthesis
gaattcttactgggatgctcttcg

SEQ ID NO: 37 - DNA, 33 bp, Artificial
  synthesis
ctgccttaggcttatgtgatctccctgagaccc

SEQ ID NO: 38 - DNA, 28 bp, Artificial
  synthesis
tctcgagttacttcctccttaatctttc

SEQ ID NO: 39 - DNA, 33 bp, Artificial
  synthesis
ctgccttaggcttatgtgatctgcctcaaaccc

SEQ ID NO: 40 - DNA, 27 bp, Artificial
  synthesis
tctcgagtcattccttacttcttaaac

SEQ ID NO: 41 - DNA, 33 bp, Artificial
  synthesis
ctgccttaggcttatacaacttgcttggattcc

SEQ ID NO: 42 - DNA, 26 bp, Artificial
  synthesis
cactcgagtcagtttcggaggtaacc

SEQ ID NO: 43 - DNA, 37 bp, Artificial
  synthesis
ctgccttaggcttatgtgatctgcctcagaaccatgg

SEQ ID NO: 44 - DNA, 26 bp, Artificial
  synthesis
ctcgagtcaagatgagcccaggtctc

SEQ ID NO: 45 - DNA, 37 bp, Artificial
  synthesis
actccttaggcttacaggacccatatgtacaagaagc

SEQ ID NO: 46 - DNA, 24 bp, Artificial
  synthesis
ctcgagttactgggatgctcttcg